(19) United States
(12) Patent Application Publication
(10) Pub. No.: US 2021/0343420 A1
(43) Pub. Date: Nov. 4, 2021

(54) SYSTEMS AND METHODS FOR PROVIDING ACCURATE PATIENT DATA CORRESPONDING WITH PROGRESSION MILESTONES FOR PROVIDING TREATMENT OPTIONS AND OUTCOME TRACKING

(71) Applicant: COTA, Inc., Boston, MA (US)

(72) Inventors: Nicholas Ritter, Huntsville, AL (US); Stephen Jakubowicz, Lancaster, PA (US); Meng Mao, Cambridge, MA (US); Michael Mulcahy, Draper, UT (US); Sudhakar Velamoor, Sharon, MA (US); Monica Matta, New York, NY (US); Ching-Kun Wang, Fort Worth, TX (US); Micha Hanson, Boston, MA (US); Scott Cady, Denver, CO (US); Tanvi Pal, Boston, MA (US)

(73) Assignee: COTA, Inc., Boston, MA (US)

(21) Appl. No.: 17/375,916

(22) Filed: Jul. 14, 2021

Related U.S. Application Data
(63) Continuation-in-part of application No. 17/146,260, filed on Jan. 11, 2021.
(60) Provisional application No. 62/958,883, filed on Jan. 9, 2020.

(51) Int. Cl. G16H 50/30; G16H 10/60; G06F 16/215

(52) U.S. Cl. CPC G16H 50/30; G06F 16/215; G16H 10/60

(57) ABSTRACT

Described herein is a system, method, and non-transitory computer-readable medium, to provide accurate patient data corresponding with diagnosis and/or progression milestones for a patient with a medical condition and/or illness. Also described herein are methods and systems for providing a graphical user interface including an interactive patient information timeline.

### RELATED APPLICATIONS

This application is a continuation-in-part of U.S. application Ser. No. 17/146,260, filed Jan. 11, 2021, which claims priority to and benefit of U.S. Provisional Patent Application No. 62/958,883, filed Jan. 9, 2020. The disclosure of both applications are hereby incorporated herein by reference in their entireties.

### BACKGROUND

Currently, systems that store and organize patient data for use in later analysis or in systems for guiding patient treatment options either store large volumes of data without determining what data is best or most accurate, or heavily rely on human interpretation to determine what data is the best or most accurate for storage. Primarily relying on unguided human interpretation to determine what data is best or most accurate complicates the data output process, introduces additional sources of error, and can result in different standards being applied to data from different patients, and different types of data being stored for different patients.

### SUMMARY

Some embodiments herein provide systems and methods for determining and providing accurate patient data associated with progression milestones or a timeline. In some embodiments, such accurate patient data is used by other systems or applications, such as a system for providing and displaying accurate and succinct patient data, a system for assigning a patient to a nodal address, a system for assisting a health care provider in providing treatment options to a patient, and/or a system for predicting a prognosis-related expected outcome.

According to one aspect, the described invention provides a method for providing accurate patient data for a patient with a medical condition and/or illness, the method including: accessing an initial set of data records associated with the patient, the initial set of data records including information regarding the patient, the patient's illness, and/or the patient's treatment; extracting a plurality of candidate facts from the accessed initial set of data records, each candidate fact represented as a data set; and categorizing each candidate fact as corresponding to an element of a plurality of elements associated with the patient, the plurality of candidate facts including more than one candidate fact corresponding to the element for at least one element in the plurality of elements. The method also includes for elements that are unchanging over time, identifying at least one best fact corresponding to each element, the identifying including: where the element has only one corresponding candidate fact, identifying the corresponding candidate fact as the best fact corresponding to the element; and where the element has at least two corresponding candidate facts, identifying at least one of the corresponding candidate facts for the element as the best fact for the element based on reduction rules specific to the element. The method also includes for each element that can change over time, associating each candidate fact corresponding to the element with a progression period corresponding to a diagnosis or progression milestone; for each element that can change over time, identifying at least one best fact for each progression period having an associated candidate fact for the element, the identifying including: where the element has only one corresponding candidate fact associated with the progression period, identifying the corresponding candidate fact as the best fact corresponding to the element for the progression period; and where the element has at least two corresponding candidate facts associated with the progression period, identifying at least one best fact corresponding to the element for the progression period from the at least two corresponding facts based on reduction rules specific to the element. The method also includes outputting data including the best facts associated with the patient.

In some embodiments of the method, for at least some of the elements that are unchanging over time, identifying the at least one best fact corresponding to the element further includes: presenting the at least one best fact as a suggested at least one best fact corresponding to the element to a user via a graphical user interface; receiving one or more of: an acceptance of the suggested at least one best fact, an identification of at least one other candidate fact that is not a suggested best fact as the at least one best fact, and a rejection of the suggested at least one best fact as a best fact; and where a rejection of the suggested at least one best fact is received, no longer identifying the suggested at least one best fact as a best fact corresponding to the element; where an acceptance of the suggested at least one best fact is received, identifying the at least one best fact as an accepted best fact; and where an identification of at least one other candidate fact that is not a suggested best fact as at least one best fact is received, identifying the at least one other candidate fact as the at least one accepted best fact; wherein outputting data regarding the best facts associated with the patient includes outputting data regarding the accepted best facts associated with the patient.

In some embodiments of the method, for at least some of the elements that can change over time, identifying at least one best fact for each progression period having an associated candidate fact for the element further includes: presenting the at least one best fact for the progression period as a suggested at least one best fact corresponding to the element; receiving one or more of: an acceptance of the suggested at least one best fact as at least one best fact; an identification of at least one other candidate fact that is not a suggested best fact as at least one best fact; and a rejection of the suggested at least one best fact as a best fact; and where a rejection of the suggested at least one best fact is received, no longer identifying the suggested at least one best fact as a best fact corresponding to the element.

In some embodiments of the method, the output data including the best facts associated with the patient includes progression output. In some embodiments of the method, the output data including the best facts associated with the patient include progression output and time series output. In some embodiments of the method, the output data including the best facts associated with the patient includes progression output, or progression output and time series output. In some embodiments of the method, the progression output is data indexed by progression period or by diagnosis and progression milestones for the patient. In some embodiments of the method, the progression output includes the best facts stored in associated concept tables, each concept table including a progression track identifier and a patient identifier. In some embodiments of the method, the time series output includes the best facts stored in associated concept tables, each associated concept table indexed by a function of time elapsed between a start date and time associated with the best fact in the associated concept table.

In some embodiments, the method further includes: determining, based on at least some of the candidate facts, one or more progression periods, each progression period corresponding to a period of time beginning at diagnosis or at a progression of the medical condition or illness and ending at a next progression, at the present time, or at death; and assigning each candidate fact to a progression period. In some embodiments, the method further includes: presenting the determined one or more progression periods to a user via a graphical user interface as suggested progression periods; receiving input from a user including one or more of: an acceptance of at least one of the one or more suggested progression periods, an adjustment of a start time or an end time of at least one of the one or more suggested progression periods, an addition of a new progression period, or merging of at least some of the one or more of the suggested progression periods into a single progression time period; and adjusting the one or more progression periods based on the received input, wherein each candidate fact is assigned to a progression time period after the adjusting. In some embodiments, the progressions correspond to one or more of: a physician's identification that the patient's disease or condition has progressed; a measured growth of a tumor of the patient; an indication that the patient's disease has spread and become metastatic; an indication that the patient's disease or medical condition has not responded to a course of treatment and a physician has decided to switch to a different course of treatment; or an indication that the patient has experienced a relapse in disease or the medical condition.

In some embodiments, for each element that can change over time, the associating of each candidate fact corresponding to the element with a progression period is based on time windowing.

In some embodiments, the method further includes: accessing a new set of data records; extracting additional candidate facts, each of the additional candidate facts corresponding to an element of the plurality of elements associated with the patient; and determining one or more best facts corresponding to the each element of the plurality of elements based on the plurality of candidate facts extracted from the initial set of data records and the additional candidate facts extracted from the new set of data records.

In some embodiments, the method also includes de-duplicating the plurality of candidate facts by, for each element in the plurality of elements, removing each duplicative candidate fact.

In some embodiments, the method also includes: deriving a candidate fact for at least one element of the plurality of elements associated with the patient based on one or more of the candidate facts extracted from the data and one or more medical rules.

In some embodiments, the data records associated with a patient are abstracted data records.

In some embodiments, for at least one of the elements, the reduction rules include a rule to identify at least one candidate fact as a best overall fact for an element based the candidate fact including the most amount of data as compared to other candidate facts corresponding to the same element.

In some embodiments, for at least one of the elements, the reduction rules include a rule to discard a candidate fact that is duplicative of and identical to another candidate fact corresponding to an element for a progression period.

In some embodiments, for at least some of the elements, the reduction rules include a rule to identify a candidate fact as a best fact based, at least in part, on the candidate fact being the most frequently occurring as compared to other candidate facts corresponding to the same element.

In some embodiments, the method further includes: for at least one progression period, generating a nodal address for the progression period for the patient based on the output data. In some embodiments, the method further includes: providing predetermined treatment plan information to a health care provider of the patient for facilitation of treatment decisions, the predetermined treatment plan information based on the nodal address for the progression period assigned to the patient. In some embodiments, the method further includes: determining a prognosis-related expected outcome with respect to occurrence of the defined end point event for the patient based on the nodal address for the progression period assigned to the patient. In some embodiments, the generated nodal address is a refined nodal address.

According to one aspect, the described invention provides a system for providing accurate patient data for a patient with a medical condition and/or illness, the method including: one or more data repositories; and a computing system in communication with the one or more data repositories and configured to execute instructions that when executed cause the computing system to: access, from the one or more data repositories, an initial set of data records associated with the patient, the initial set of data records including information regarding the patient, the patient's illness, and/or the patient's treatment; extract a plurality of candidate facts from the accessed initial set of data records, each candidate fact represented as a data set; categorize each candidate fact as corresponding to an element of a plurality of elements associated with the patient, the plurality of candidate facts including more than one candidate fact corresponding to the element for at least one element in the plurality of elements. The instructions further causing the computing system to: for elements that are unchanging over time, identify at least one best fact corresponding to each element, the identification including: where the element has only one corresponding candidate fact, identifying the corresponding candidate fact as the best fact; and where the element has at least two corresponding candidate facts, identifying at least one of the corresponding candidate facts for the element as the best fact for the element based on reduction rules specific to the element. The instructions further causing the computing system to: for each element that can change over time, associate each candidate fact corresponding to the element with progression period corresponding to a diagnosis or progression milestone; for each element that can change over time, identify at least one best fact for each progression period having an associated candidate fact for the element, the identification including: where the element has only one corresponding candidate fact associated with the milestone, identifying the corresponding candidate fact as the best fact corresponding to the element for the progression period; and where the element has at least two corresponding candidate facts associated with progression period, identifying at least one best fact corresponding to the element for the milestone from the at least two corresponding candidate facts based on reduction rules specific to the element; and output data including the best facts associated with the patient.

According to one aspect, the described invention provides a non-transitory computer readable medium including program instructions for providing accurate patient data for a patient with a medical condition and/or illness, wherein execution of the program instructions by one or more processors causes the one or more processors to perform any of the methods recited or claimed herein.

According to one aspect, the described invention provides a method for providing a graphical user interface for visualizing patient data. The method includes the method displaying an interactive timeline graphically depicting information regarding a patient's medical history, the interactive timeline including a plurality of markers, each marker indicating a relevant time associated with medical information of the patient, a beginning of a period of time associated with the medical information of the patient (e.g., information in the patient's medical history or patient's medical record), or an end of a period of time associated with medical information of the patient. The interactive timeline includes a plurality of sub-timelines for different categories of medical information vertically offset and aligned in time with each other. The plurality of sub-timelines including one or more of: a treatment sub-timeline including any markers related to treatment information, a diagnosis or progression sub-timeline including any markers related to diagnosis or disease or disorder progression information, a biomarker sub-timeline including any markers related to disease or disorder biomarker test results information, a disease or disorder sub-timeline including any markers related to disease or disorder information not falling in other categories, and a patient sub-timeline including any markers related to relevant medical information not falling into other categories. The method also includes receiving a user input selecting a marker; and displaying detailed medical information associated with the marker in a window in the interactive timeline.

In some embodiments, the interactive timeline further includes one or more vertical graphical indicators, each representing diagnosis or a disease progression. In some embodiments, the interactive timeline includes one or more diagnosis or progression time periods. In some embodiments, the one or more diagnosis or progression time periods are divided by the one or more vertical graphical indicators. In some embodiments, the graphical user interface enables filtering of markers displayed the interactive timeline based on user selected criteria. In some embodiments, the user-selected criteria include a diagnosis or progression time period.

In some embodiments, a shape of a beginning marker and a shape of an ending marker indicates a degree of precision regarding a date for information associated with the marker. In some embodiments, a shape of one or more markers of the plurality of markers indicates a degree of precision regarding a date for information associated with the one or more markers.

In some embodiments, the method further comprises displaying a summary version of the full time period timeline including two or more selectable graphical indicators, the selectable graphical indicators including a beginning time period indicator and an ending time period indicator, where selection and movement of the beginning time period indicator and/or the ending time period indicator changes a time period displayed in the interactive timeline.

In some embodiments, the plurality of sub-timelines further includes one or more of: a systemic therapy sub-timeline including any markers related to systemic therapy information, a surgery sub-timeline including any markers related to surgery information, and a radiation treatment sub-timeline including any markers related to radiation treatment.

In some embodiments, markers in a first sub-timeline of the plurality of sub-timelines are depicted in a color that is different from a color of markers in a second sub-timeline of the plurality of sub-timelines. In some embodiments, a color of a window including medical information that is displayed upon selection of a marker is a same color as that of the maker selected.

In some embodiments, all medical information displayed via the interactive patient timeline is de-identified to protect patient privacy.

In some embodiments, the interactive timeline is a first interactive timeline for a first patient's medical history, and the method further comprises displaying a second interactive timeline graphically depicting information regarding a second patient's medical history for comparison with the first interactive timeline, where the second interactive timeline is aligned with the first interactive timeline based on a time of diagnosis or a disease progression for both the first patient and the second patient, and where all medical information in the first interactive timeline and the second interactive timeline is de-identified.

In some embodiments, one or more time periods associated with medical information are graphically displayed with a beginning marker and an ending marker and a graphical indication of span between the beginning marker and the ending marker.

According to one aspect, the described invention provides a non-transitory computer readable medium including program instructions for providing a graphical user interface including an interactive patient timeline, wherein execution of the program instructions by one or more processors causes the one or more processors to perform any of the methods recited or claimed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. In the drawing figures, which are not to scale, and where like reference numerals indicate like elements throughout the several views:

FIG. 1 illustrates a network diagram to provide accurate patient data using an Enrichment Layer (EL) module in accordance with an embodiment of the present disclosure;

FIG. 2 schematically illustrates the EL in Relation to Abstraction, Patient Data, and Products in accordance with an exemplary embodiment;

FIG. 3 schematically illustrates an exemplary reduction of candidate facts in accordance with an exemplary embodiment;

FIG. 4 schematically illustrates an exemplary reduction of candidate facts based on medically-based reduction rules in accordance with an exemplary embodiment;

FIG. 5 schematically illustrates exemplary calculating and/or deriving of candidate facts in accordance with an exemplary embodiment;

FIG. 6 illustrates an EL workflow in accordance with an exemplary embodiment;

FIG. 7 schematically illustrates generation of a nodal address from the EL output in accordance with an exemplary embodiment;

FIG. 8 schematically illustrates a read-only database permission model in accordance with an exemplary embodiment;

FIG. 9 schematically illustrates an exemplary EL kernel 1002 in accordance with an exemplary embodiment;

FIG. 10 schematically illustrates a Shape used for processing input data and candidate facts in accordance with some embodiments;

FIG. 11 schematically illustrates attributes associated with a Shape in accordance with an exemplary embodiment;

FIG. 12 is a flowchart illustrating a process of identifying a best fact for a corresponding element;

FIG. 13 is a flowchart depicting a process of determining a best fact in response to receiving additional data;

FIG. 14 is a flowchart depicting a process for conflict resolution or escalation in accordance with an exemplary embodiment;

FIG. 15 is a screen shot of a user interface for acceptance or verification of suggested best facts and progression periods in accordance with some embodiments;

FIG. 16 schematically illustrates an architecture for a system in accordance with some embodiments;

FIG. 17 depicts one example of a schematic diagram illustrating a client device in accordance with an embodiment of the present disclosure;

FIG. 18 is a block diagram illustrating an internal architecture of a computer in accordance with an embodiment of the present disclosure;

FIG. 19 depicts a graphical user interface including an interactive patient timeline with windows including medical information for a patient displayed for selected markers in an initial diagnosis time period accordance with some embodiments;

FIG. 20 depicts the graphical user interface including the interactive patient timeline of FIG. 19 with windows including medical information for the patient displayed for selected markers in a diagnosis of metastatic cancer (e.g. progression to metastatic cancer) time period in accordance with some embodiments;

FIG. 20A depicts windows including medical information that are displayed overlaid on the interactive patient timeline of FIG. 20 when corresponding markers indicated with numbers 1-11 are selected by a user in accordance with some embodiments;

FIG. 21 depicts the graphical user interface including the interactive patient timeline of FIG. 19 displaying a zoomed-in or enlarged portion of the timeline based on a user selection of beginning and ending times in a summary timeline for time period from diagnosis of metastatic cancer to a first metastatic disease progression of the cancer with windows including medical information for the patient displayed for first set of selected markers in that time period in accordance with some embodiments;

FIG. 22 depicts the graphical user interface including the interactive patient timeline of FIG. 19 displaying the zoomed-in or enlarged portion of the timeline and the interactive patient timeline displaying windows with medical information corresponding to a second set of selected markers in accordance with some embodiments;

FIG. 23 depicts the graphical user interface including the interactive patient timeline of FIG. 19 displaying the zoomed-in or enlarged portion of the timeline and the interactive patient timeline displaying windows with medical information corresponding to a third set of selected markers in accordance with some embodiments;

FIG. 24 depicts the graphical user interface including the interactive patient timeline of FIG. 19 displaying the zoomed-in or enlarged portion of the timeline and the interactive patient timeline displaying a window with medical information corresponding to a later selected marker in accordance with some embodiments;

FIG. 25 illustrates an example interface showing summary patient information in accordance with an exemplary embodiment;

FIG. 26 illustrates an example interface displaying patient information for an institution in accordance with an exemplary embodiment; and

FIG. 27 schematically depicts a system and method that incorporates best fay t enrichment for analytics in accordance with an exemplary embodiments.

### DESCRIPTION OF EMBODIMENTS

Glossary of Terms

The term “fluorescence in situ hybridization” (“FISH”) as used herein refers to a laboratory method used to look at genes or chromosomes in cells and tissues. Pieces of DNA that contain a fluorescent dye are made in the laboratory and added to a cell or tissue sample. When these pieces of DNA bind to certain genes or areas on chromosomes in the sample, they light up when viewed under a microscope with a special light. FISH can be used to identify where a specific gene is located on a chromosome, how many copies of the gene are present, and any chromosomal abnormalities.

The term “immunohistochemistry” or “IHC” testing as used herein refers to a special staining process performed on fresh or frozen cancer tissue removed during biopsy that uses antibodies to identify specific proteins in tissue sections.

The term “next generation sequencing” or NGS” as used herein refers to a sequencing method that provides a comprehensive view of a tumor's genomic profile and can detect multiple mutations present at very low levels within the tumor.

The term “polymerase chain reaction” (“PCR) as used herein refers to a laboratory method used to make many copies of a specific piece of DNA from a sample that contains very tiny amounts of that DNA. PCR allows these pieces of DNA to be amplified so they can be detected. PCR may be used to look for certain changes in a gene or chromosome, which may help find and diagnose a genetic condition or a disease, such as cancer. It may also be used to look at pieces of the DNA of certain bacteria, viruses, or other microorganisms to help diagnose an infection.

Embodiments are now discussed in more detail referring to the drawings that accompany the present application. In the accompanying drawings, like and/or corresponding elements are referred to by like reference numbers.

Some embodiments described herein include a system, method, and/or non-transitory computer-readable medium for providing accurate patient data for a patient with a medical condition and/or illness. In some embodiments, at least a portion of the accurate patient data includes facts associated with progression periods corresponding to diagnosis or progression milestones. In some embodiments, at least a portion of the accurate patient data includes facts associated with a timeline. In some embodiments, the accurate patient data can be provided to a system configured to identify treatment options for the patient, a system configured to evaluate treatment of the patient, or a system configured to determine an expected outcome of the patient. Some embodiments improve the efficiency of the system or method by enabling other systems to operate on only the most accurate data and only store the most accurate data. Further, some embodiments improve the efficiency in storage by storing only the most accurate data, instead of storing all data.

In some embodiments, a method or system employs an enrichment layer module that implements an Enrichment Layer (EL) to determine or assist in determining accurate patient data. In some embodiments, the system or method receives or accesses potential candidate facts which correspond with elements associated with patients with the medical condition and/or illness. The elements can be associated with the patient or the medical condition and/or illness. For example, the elements can be name, age, prognosis, treatments, and other information associated with the patient or medical condition and/or illness. In some embodiments, the EL can identify the best (or most accurate) fact or facts from the candidate facts for an element associated with the patient, by deriving, calculating, and/or reducing the candidate facts. In some embodiments, the EL can identify at least one suggested best or most accurate fact subject to acceptance or verification by a user. In some embodiments, the at least one suggested best or most accurate fact determined by the EL is presented via a graphical user interface for acceptance or verification. In some embodiments, for some elements, the EL identifies the best fact and for other elements, the EL provides a suggestion for the best fact, which can be accepted or overridden.

In some embodiments, the EL can evaluate end to end patient information covering the course of the patient's medical history from diagnosis through multiple points up until death and identifies the most accurate facts, or suggested most accurate facts, regarding the patient from the patient information. In some embodiments, the system or method generates a progression and/or timeline based output representing the identified best facts. The best facts corresponding to each element associated with the patient can represent a complete and current view of the patient's medical condition and illness history. In this regard, in some embodiments, the method or system largely or completely eliminates the manual process of deciding which facts are accurate and which are incomplete by efficiently collecting data, and automatically deriving, calculating, and reducing candidate facts to identify the best fact corresponding to the element.

In some embodiments, the method or system can evaluate end to end patient information covering the course of the patient's medical history from diagnosis through multiple points up until death and generate suggestions for the most accurate facts regarding the patient from the patient information, subject to acceptance or verification. In some embodiments, the system or method generates a progression and/or timeline based output representing the identified best facts after acceptance or verification. The best facts corresponding to each element associated with the patient can represent a complete and current view of the patient's medical condition and illness history. In this regard, in some embodiments, EL reduces unpredictability in a manual process of determining which facts are accurate and which are incomplete by efficiently collecting data, and automatically deriving, calculating, and reducing candidate facts to identify the suggested best facts in a reproducible and predictable manner.

In some embodiments, the method or system for identifying the best facts as described in the present disclosure simplifies clinical data querying and exploration. Traditionally, oncology centers that invest in data collection, tracking, and analysis must invest in IT infrastructure and budget for a team to field data requests. Data requests serve to help the oncology centers to evaluate different aspects of their practice—from patient populations for clinical trial feasibility to providing data for care delivery quality improvement initiatives. For each data request, the turnaround time can range from a few weeks to a few days depending on the data request, an institution's sophistication and investment in data infrastructure, and a data analytics team's competencies. In some embodiments, the best facts identification as described in the present disclosure may measurably reduce the time needed to fulfill data requests for an institution. Furthermore, the best fact identification can serve as the foundation for technologies that seek to provide real-time data querying and exploration where data requests can be fulfilled instantaneously (i.e., within seconds).

Various embodiments are disclosed herein; however, it is to be understood that the disclosed embodiments and user interfaces as shown are merely illustrative of the disclosure that can be embodied in various forms. In addition, each of the examples given in connection with the various embodiments is intended to be illustrative, and not restrictive. Therefore, specific structural and functional details disclosed herein are not to be interpreted as limiting, but merely as a representative basis for teaching one skilled in the art to variously employ the disclosed embodiments.

Embodiments are described below with reference to block diagrams and operational illustrations of methods and systems. It is understood that each block of the block diagrams or operational illustrations, and combinations of blocks in the block diagrams or operational illustrations, can be implemented by means of analog or digital hardware and computer program instructions. These computer program instructions can be provided to one or more processors of a general purpose computer, special purpose computer, ASIC, or other programmable data processing apparatus, such that the instructions, which execute via one or more processors of the computer or other programmable data processing apparatus, implements the functions / acts specified in the block diagrams or operational block or blocks.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved. Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Although described herein primarily with respect to cancer conditions, the described methods and systems can be for patient data corresponding to any progressive clinical condition (e.g., cardiovascular disease, metabolic disease (diabetes), immune mediated diseases (e.g., lupus, rheumatoid arthritis), organ transplantation, neurodegenerative disorders, pulmonary diseases, infectious diseases, hepatic disorders). A practitioner would know the parameters of each such condition. In some embodiments, the methods and systems are specific to cancer conditions.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

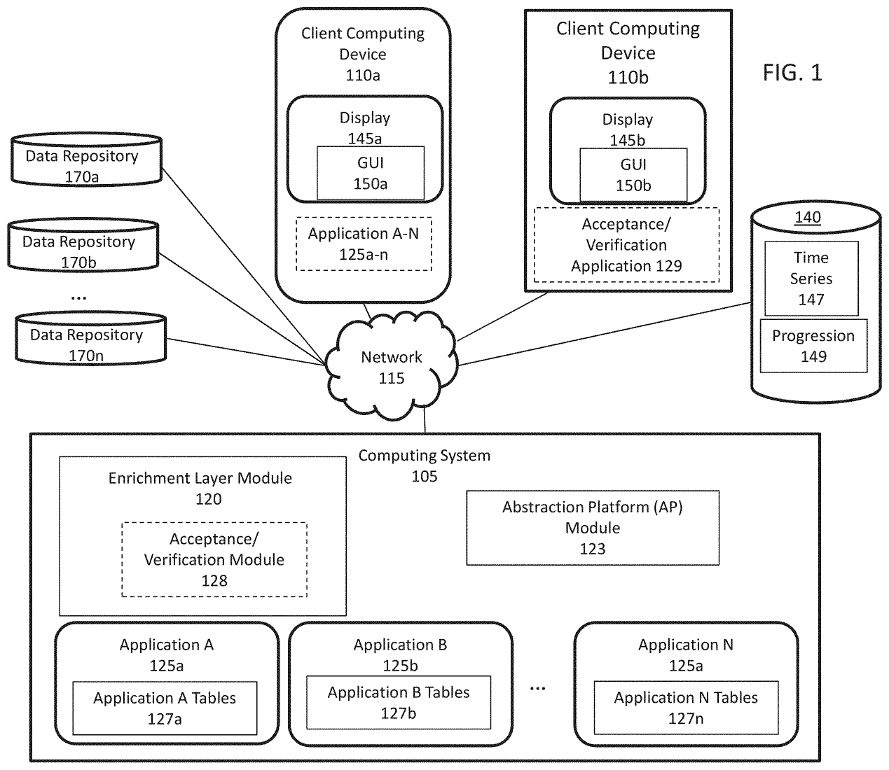

FIG. 1 schematically depicts a network diagram of computing systems, devices, networks and databases that could be employed in connection with some embodiments described herein. The depicted network diagram shows a computing system 105 communicating with client computing devices 110a, 110b, databases 140, and data repositories 170a-b over network 115. The computing system 105 can host and execute an abstraction 123 module, application or platform, an EL module 125, and applications A-N 127a-n. It can be appreciated that the each of the abstraction module 123, EL module 125, and applications A-N 125a-n can be executed on the same or separate computing systems. The computing system 105 can further communicate with disparate data repositories 170a-n over the network 115, to retrieve data.

The computing system 105 can host one or more applications configured to interact with one or more components and/or facilitate access to the content of the databases 140 and data repositories 170a-n. The databases 140 and data repositories 170a-n may store information/data, as described herein. For example, the databases 140 can include a time series database 147 and a progression database 149. The time series database 147 can store identified accurate patient data output based on a time series model. The progression database 149 can store identified accurate patient data output from the EL module 120 for progression periods corresponding to diagnosis or progression milestones of a patient's disease and/or medical condition. The data repositories 170a-n can store patient information and medical information. The databases 140 can be located at one or more geographically distributed locations from the computing system 105. Alternatively, the databases 140 can be located at the same geographic location as the computing system 105.

The computing system 105 can execute the EL module to identify accurate patient data as described herein. In one embodiment, the accurate patient data can be provided to or accessed by the applications A-N 125a-n. The applications A-N 125a-n can store the patient data in respective application tables 127a-n of each application A-N 125a-n. An instance of one or more of each of the applications A-N 125a-n can be executed on a client computing device 110a. Each of the applications A-N 125a-n can provide a user interface (e.g., a graphical user interface 150a to be rendered on the display 145a of the client computing device 110a). The term “UI” refers to a user interface, which is the point of human-computer interaction and communication in a device or system. This can include display screens, keyboards, a mouse and the appearance of a desktop. A user interface can also refer to a way through which a user interacts with an application or a website. Each of the applications A-N 125a-n can be configured to output the identified accurate patient data to be rendered on the graphical user interface 150a rendered on the display 145. Alternatively or in addition, the identified accurate patient data can be used by any of the applications A-N 125a-n to generate an output to be rendered on the graphical user interface 150a rendered on the display 145a of the client computing device 110a. Alternatively, or in addition, the EL module 120 can store the identified accurate patient data in a time series database 147 and/or a progression database 149.

In some embodiments, an acceptance/verification module 128 is used in the process of identifying accurate patient data. In some embodiments, the acceptance/verification module 126 is executed, at least in part, as an acceptance/verification application 129 on a client computing device 110b different from a client computing device 110a that hosts any of Applications A-N 125a-n. In some embodiments, at least some aspects of acceptance and verification may be executed by an abstraction platform. In some embodiments, a graphical user interface 150b of the client computing device 110b is used to receive input from a user for acceptance or verification of some or all of the identified best data. In other embodiments, the acceptance/verification module 126 is executed wholly by the computing system 105 and receives input from a user for acceptance or verification of some or all of the identified best data from a graphical user interface of the computing system 105.

The computing system 105 may generate and/or serve content such as web pages, for example, to be displayed by a browser (not shown) of client computing device 110a, 110b over network 115 such as the Internet. In some embodiments, the one or more of the applications A-N 125a-n or the acceptance/verification application 129 is executed at least in part as a web page (or part of a web page) and is therefore accessed by a user of the client computing device 110a, 110b via a web browser. In some embodiments, one or more of the applications A-N 125a-n or the acceptance/verification application 129 is a software application, such as a mobile “app”, that can be downloaded to the client computing device 110a, 110b from the computing system 105. In some embodiments, one or more of the applications A-N 125a-n or the acceptance/verification application 129 provides a graphical user interface (GUI) 150a, 150b for enabling the functionality described herein, when executed on the client computing device 110a. 110b.

A computing device embodied fully or in part as computing system 105 and/or client computing device 1101, 110b may be capable of sending or receiving signals, such as via a wired or wireless network, or may be capable of processing or storing signals, such as in memory as physical memory states. Devices and systems capable of operating as computing system 105 include, but are not limited to, as examples, one or more of dedicated rack-mounted servers, desktop computers, laptop computers, set top boxes, integrated devices combining various features, such as two or more features of the foregoing devices, or the like. Embodiments of computing system 105 may vary widely in configuration or capabilities, but generally may include one or more central processing units and memory. Computing system 105 may also include one or more mass storage devices, one or more power supplies, one or more wired or wireless network interfaces, one or more input/output interfaces, or one or more operating systems, such as Windows® Server, Mac® OS X®, Unix®, Linux®, FreeBSD®, or the like. Computing system 105 may include multiple different computing devices. Computing system 105 may include multiple computing devices that are networked with each other. Computing system 105 may include networks of processors or may employ networks of remote processors for processing (e.g., cloud computing). Some aspects may be implemented, at least in part, via a cloud container engine.

The computing system 105 may include a device that includes a configuration to provide content via a network to another device. The computing system 105 may further provide a variety of services that include, but are not limited to, web services, third-party services, audio services, video services, email services, instant messaging (IM) services, SMS services, MMS services, FTP services, voice over IP (VOIP) services, calendaring services, photo services, or the like. Examples of content may include text, images, audio, video, or the like, which may be processed in the form of physical signals, such as electrical signals, for example, or may be stored in memory, as physical states, for example. Examples of devices that may operate as or be included in computing system 105 include desktop computers, multiprocessor systems, microprocessor-type or programmable consumer electronics, etc.

A network may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine readable media, for example. A network may include the Internet, one or more local area networks (LANs), one or more wide area networks (WANs), wire-line type connections, wireless type connections, or any combination thereof. Likewise, sub-networks, such as may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

A communication link or channel may include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art. Furthermore, a computing device or other related electronic devices may be remotely coupled to a network, such as via a telephone line or link, for example.

A wireless network may couple client devices with a network 115. A wireless network 115 may employ stand-alone ad-hoc networks, mesh networks, Wireless LAN (WLAN) networks, cellular networks, or the like. A wireless network 115 may further include a system of terminals, gateways, routers, or the like coupled by wireless radio links, or the like, which may move freely, randomly or organize themselves arbitrarily, such that network topology may change, at times even rapidly. A wireless network 115 may further employ a plurality of network access technologies, including Long Term Evolution (LTE), WLAN, Wireless Router (WR) mesh, or 2nd, 3rd, 4th, 5th, or 6th generation (2G, 3G, 4G, 5G, 6G) cellular technology, or the like. Network access technologies may enable wide area coverage for devices, such as client devices with varying degrees of mobility, for example.

For example, a network 115 may enable RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, or the like. A wireless network may include virtually any type of wireless communication mechanism by which signals may be communicated between devices, such as a client device or a computing device, between or within a network, or the like.

In one embodiment and as described herein, the client computing device 110 is a smartphone. In another embodiment, the client computing device 110 is a tablet. The client computing device 110 can also be a computer, a set-top box, a smart TV, or any other computing device.

In one embodiment, the abstraction module 123, EL module 120, acceptance/verification module 128, and/or applications A-N 125a-n may be implemented in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs). Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

In one embodiment, the client computing device 110a can be operated by a user. In some embodiments users may be patients, health care provider systems, payers (e.g., insurance companies), and medical professionals. The patients, health care provider systems, medical professionals, or insurance company can execute an instance of the applications A-N 125a-n on the client computing device 110a to interface with the computing system 105. The applications A-N 125a-n can render a GUI 150a on the display 145a. It can be appreciated, that, in some embodiments, the GUI 150 can be different for each application A-N 125a-n and/or type of user.

In some embodiments, the client computing device 110b is used to accept input from a user to accept or verify an identified best fact and/or a progression time period. In some embodiments, the user can execute an instance of the acceptance/verification application 129 on the client computing device 110b to interface with the computing system 105. In some embodiments, the acceptance/verification application 129 renders a GUI 150b on a display 145b of the client computing device 110b. In other embodiments, the acceptance/verification module 128 may cause a GUI to be rendered on a display of the computing system 105 itself In some embodiments, a user that accepts or verifies an identified best fact and/or a progression time period may be such a user may be a person trained or qualified to evaluate patient records.

In some embodiments, a user interface may include a voice user interface (VUI), e.g., (ALEXA voice service by Amazon).

In one embodiment, the abstraction module 123 can access data associated with a patient from the data repositories 170a-n. The data can include, but is not limited to, identification information associated with the patient, health care providers, information associated with a patient's illness, information associated with a patient's medical condition, and/or information associated with the patient's treatment. The abstraction module 123 can abstract the data retrieved from the data repositories 170a-n into candidate facts associated with the patient.

In some embodiments, the candidate facts are transmitted to and/or accessed by the EL module 120. In some embodiments, the abstracted data is stored in a time series model from which the EL module 120 pulls the data. In some embodiments, the EL module is configured to categorize each candidate fact as corresponding to an element. For one or more elements, multiple candidate facts correspond with the same element. The EL module 120 is configured to reduce, derive, and/or calculate the candidate facts to identify at least one best fact out of the candidate facts corresponding to each element associated with the patient. In some embodiments, more than one fact may be identified as the best fact out of the candidate facts for the element, in which case the system may provide information regarding the more than one fact identified as the best fact to a conflict resolution system or module for selection of a single best fact for the element. In some embodiments, the EL module outputs data regarding the best facts for the elements.

In some embodiments, identification of the at least one best fact out of the candidate facts includes presenting the identified at least one best fact as a suggested at least one best fact to a user via a graphical user interface, and receiving one or more of: an acceptance of the suggested at least one best fact; an identification of at least one other candidate fact that is not a suggested best fact as at least one best fact; or a rejection of the suggested at least one best fact as a best fact. Where a rejection of the suggested at least one best fact is received, the suggested at least one best fact is no longer identified as a best fact corresponding to the element. Where an identification of at least one other candidate fact that is not a suggested best fact as at least one best fact is received, the at least one other candidate fact is identified as the at least one accepted best fact. In such an embodiment, outputting data regarding the best facts associated with the patient is outputting data regarding the accepted best facts associated with the patient. In some embodiments, the presentation of the identified at least one best fact out of the candidate facts as a suggested at least one best fact to a user via a graphical user interface and receiving input from the user in response to determine at least one accepted best fact is referred to herein as “enrichment”.

In some embodiments, the system can output the identified best facts for the elements associated with the patient, which may be accepted best facts in some embodiments, as progression data. In some embodiments, the progression data is indexed by a progression period with which the best fact is associated. In some embodiments, the system can output the identified best facts for the elements associated with the patient, which may be accepted best facts, as time series data. In some embodiments, the “time series data” is indexed by a time (e.g., number of days) elapsed since diagnosis of the patient. In some embodiments, the system can output the identified best facts for the elements associated with the patient as progression data and as time series data.

The identified best facts for each element associated with the patient, which can be accepted best fact data in some embodiments, can be output as progression data for progression-based analysis or progression-based comparison with data for other patients, or the system can generate a progression output that associates events with progression periods. The term “progression” as used herein is meant to refer to the course of a medical condition or disease, such as cancer, as it becomes worse or relapses in the body. Progression periods are time periods that are determined by milestones in the patient's experience with the disease or medical condition. The term “milestones” as used herein includes the initial date of diagnosis; and any subsequent progressions of the medical condition or illness. In some embodiments, progressions of the medical condition or illness correspond to one or more of: a physician's identification that the patient's disease or condition has progressed; a growth of a tumor of the patient; an indication that the patient's disease has spread and become metastatic; an indication that the patient's disease or medical condition has not responded to a course of treatment and a physician has decided to switch to a different course of treatment; or an indication that the patient has experienced a relapse in disease or the medical condition. The term “relapse” as used herein refers to the return of a disease or the signs and symptoms of a disease after a period of improvement.

The window of time beginning from one milestone and up until the next milestone is considered a “progression period”, and all events that occur within each window are considered part of that progression period. This may be described as each progression period corresponding to a period of time beginning at diagnosis or at a progression of the medical condition or illness extending up until the next progression, the present time, or death, whichever occurs first. For example, the time between the initial date of diagnosis and the date of the first time the patient's disease progressed could be called “progression period 0”, and any candidate facts associated with a chemotherapy treatment within that time window would be included in “progression period 0” along with all other events that occurred in the same time window.

In some embodiments, the EL module determines one or more progression periods based on at least some of the candidate facts and assigns each candidate fact to a progression period. In some embodiments, the determined one or more progression periods are presented to a user via a GUI (e.g., GU150b) as suggested progression periods. In some embodiments, input is received from a user including one or more of: an acceptance of at least one of the one or more suggested progression periods; an adjustment of a start time or an end time of at least one of the one or more suggested progression periods; an addition of a new progression period; or merging of at least some of the one or more of the suggested progression periods into a single progression time period. In some embodiments, the one or more progression periods are adjusted based on the received input, and each candidate fact is assigned to a progression time period after the adjusting.

In some embodiments, the progression output includes separate tables for each distinct concept, with each element being associated with one or more of the concepts. In some embodiments, the concept may be represented by a Shape as described below. Concepts include, without limitation, overall stage, lymphovascular invasion, race, etc. In some embodiments, once EL has determined the progressions and associated progression periods for a patient, a unique hash called a progression track ID is generated for each progression period. In some embodiments, once the “best facts” are determined, each is saved into its associated concept table with its corresponding progression track id and patient id as unique identifiers. In order to construct the full record of a patient, one could query each of the concept tables using that patient's progression track ids. The progression output tables can be output or pushed downstream to be used for analytics based on progression or nodal address generation.

As noted above, in some embodiments, the abstraction platform “AP” populates times series tables with all abstracted facts prior to selection of the best facts. The time series data output from EL is different from the time series data from the AP because the best facts from the progression model that employs the progression periods are used to populate the times series tables output from the EL, unlike times series data from the AP, which includes all abstracted data.

In some embodiments, the structure of the time series output data is the same for the AP output and the EL output. In some embodiments, time series tables mirror progression based tables in that each concept is represented in its own table. Rather than being associated with a progression time window, the time series data is represented by its index from the date of initial diagnosis, e.g., each event is assigned an index which is a function of the difference in days between the event date and the date of initial diagnosis.

In some embodiments, the EL time series output data and/or the progression output data can include information regarding TNM, which is a system employing information derived from the data records to describe the amount and spread of cancer in a patient's body. T represents the size of the tumor and any spread of cancer into nearby tissue; N represents spread of cancer to nearby lymph nodes; and M represents metastasis (spread of cancer to other parts of the body).

The system (e.g., the EL module 120) can store the identified best facts for each element associated with a patient output as progression data in the progression database 149. In some embodiments, alternatively, or in addition, the system (e.g., the EL module 120) can store the identified best facts for each element associated with the patient output as time series data in the time series database 147. The applications A-N 125a-n can access the identified best facts for each element associated with the patient from the time series database 147 or the progression database 149. In some embodiments, the EL module 120 can directly output the best facts for each element associated with the patient to the applications A-N 125a-n.

As used herein, the term “enrichment” as used herein refers to a enrichment layer component that assists a user in defining progressions and nodal address best facts in some embodiments, or that defines progressions and nodal address best facts in some embodiments.

The term “analytics schema” as used herein refers to a layer on top of abstracted and enriched data where calculations are stored and application specific tables are constructed in some embodiments.

In some embodiments, the analytics schema is used to store additional calculated or derived information that is calculated or derived based on the candidate facts or the best facts. For example, in some embodiments, a therapy intent is calculated based on candidate facts or best facts and can be stored in the analytics schema. In other embodiments, additional calculated or derived information could be stored elsewhere. Data generated by the EL is referred to as enriched data.

In some embodiments, the analytics schema stores the frequency and distribution of treatment changes. The analytics schema can be used by one or more applications, such as Real-World Analytics (RWA), and a nodal address generation module. The aforementioned applications can re-present the progression data output. Alternatively, the aforementioned applications can use the progression data output to generate further data.

For embodiments in which the output includes times series data, the output can be used to generate time series data dumps. The time series data dumps can be used for increased scrutiny of the patient timeline and events. As a non-limiting example, while a health care provider user that receives output from one of the applications may only want to see summary information about their own patients, or macro information across many patients, a pharmaceutical industry user that receives output from one of the applications may want to see each and every instance that a lab value was measured. These disparate needs by different users require different approaches to assembling patient data that corresponds to the patient's story.

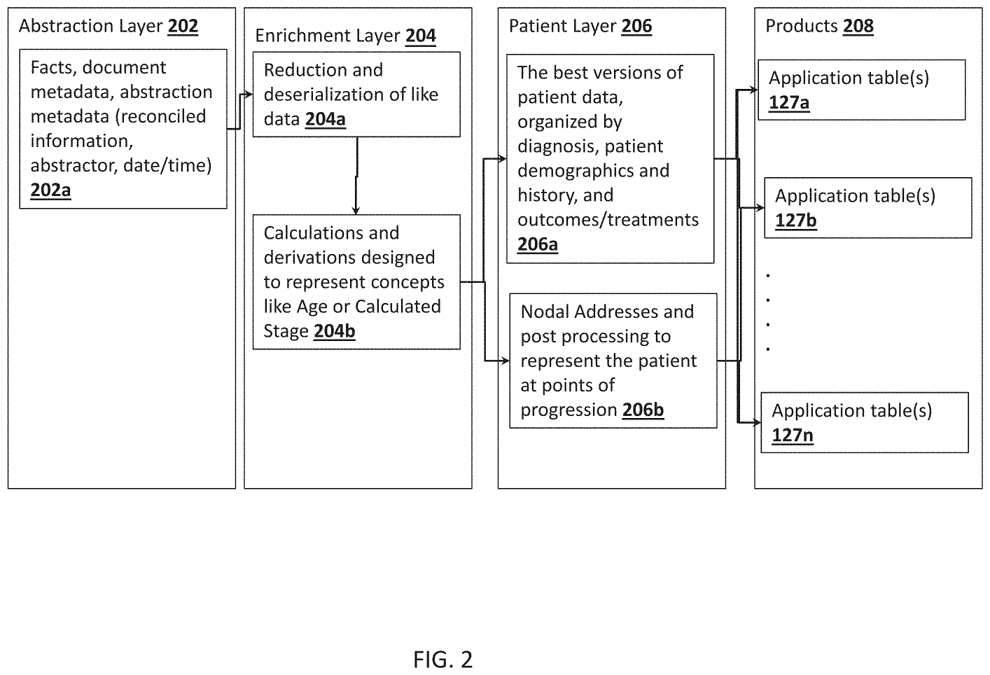

FIG. 2 illustrates the EL in Relation to an Abstraction layer 202, a Patient Data layer 204, and Products or applications 208 in accordance with an exemplary embodiment. In one embodiment, the abstraction layer 202 can execute the abstraction platform module or application 123. The abstraction module 123 can be an abstraction platform. The term “Abstraction Platform” (AP) as used herein refers to a clinical abstraction platform. Over time the abstraction layer 202 can collect, access, and/or retrieve facts, document metadata, and abstraction metadata associated with a patient from various data repositories 170a-n (202a). The data associated with the patient can be abstracted in the abstraction layer 202 to generate candidate facts corresponding with one or more elements associated with a patient. An element can be personal identification information, a medical concept, treatment information, information associated with a medical condition and/or illness, or other information associated with the patient. Some elements may correspond to a fact that would be treated as not changing over time, such as name and/or other identification information. Other elements may be treated as elements that can change over time, such as a prognosis of an illness, a medical condition of the patient, treatments provided, and/or age. Multiple candidate facts can correspond to a single element. Each of the candidate facts associated with the patient can be transmitted to or accessed by the EL 204.

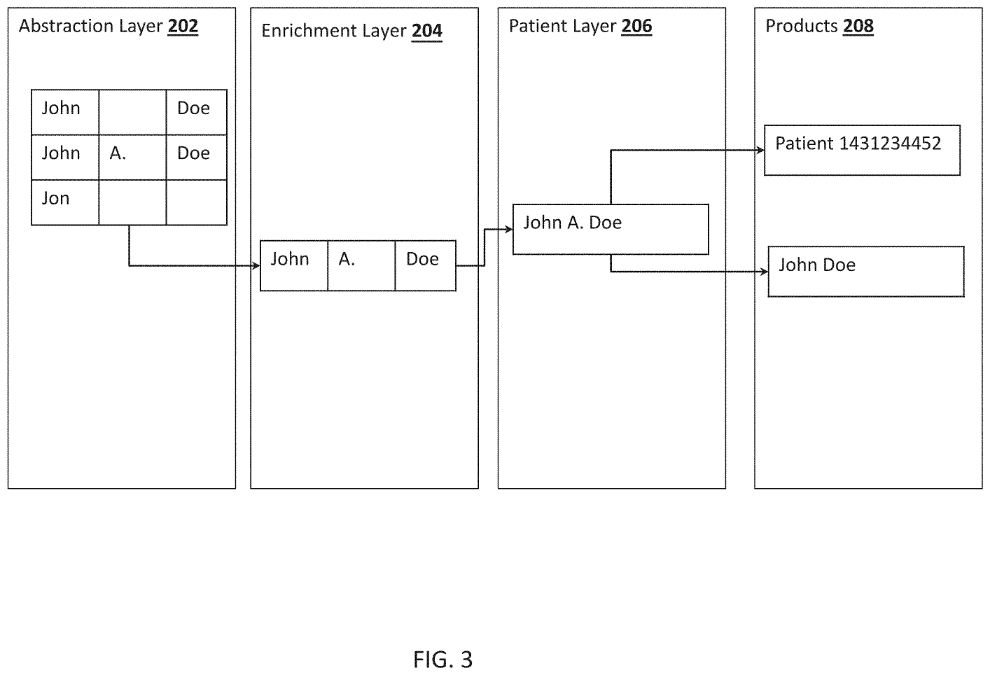

The EL layer 204 can execute the EL module 120. The EL module 120 is configured to receive the candidate facts corresponding to the elements associated with a patient. The EL module 120 can identify the best fact for a specific element from the candidate facts corresponding element based on reduction rules. The reduction rules can include processes such as de-duplication and de-serialization (204a). In some embodiments, the EL module 120 can de-duplicate and de-serialize the candidate facts to eliminate candidate facts corresponding to elements that are duplicative or incorrect. For example, the de-duplicate process can remove any redundant candidate facts. The de-serialization process determines at least one best fact from the candidate facts corresponding to the element. The reduction rules will be described in greater detail with respect to FIG. 3.

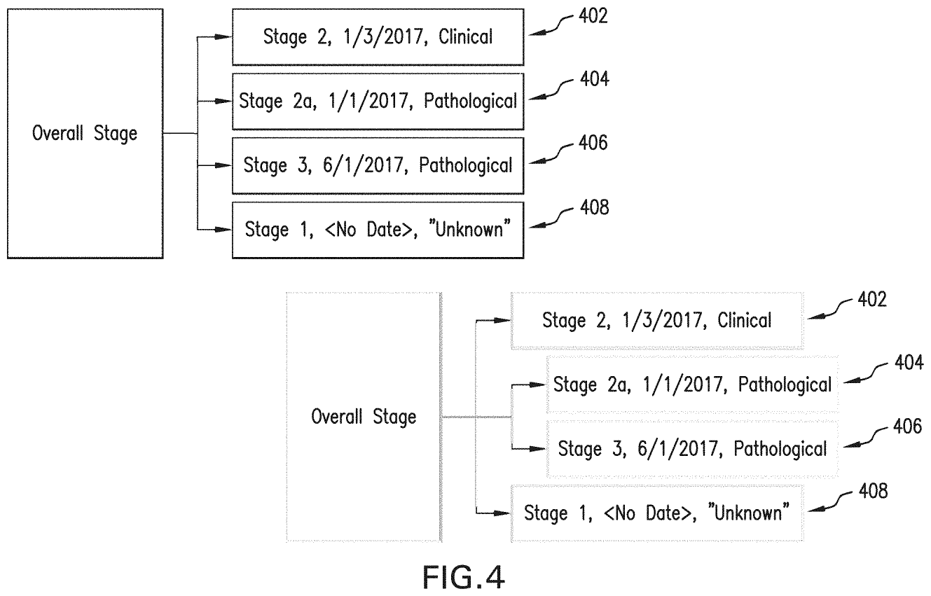

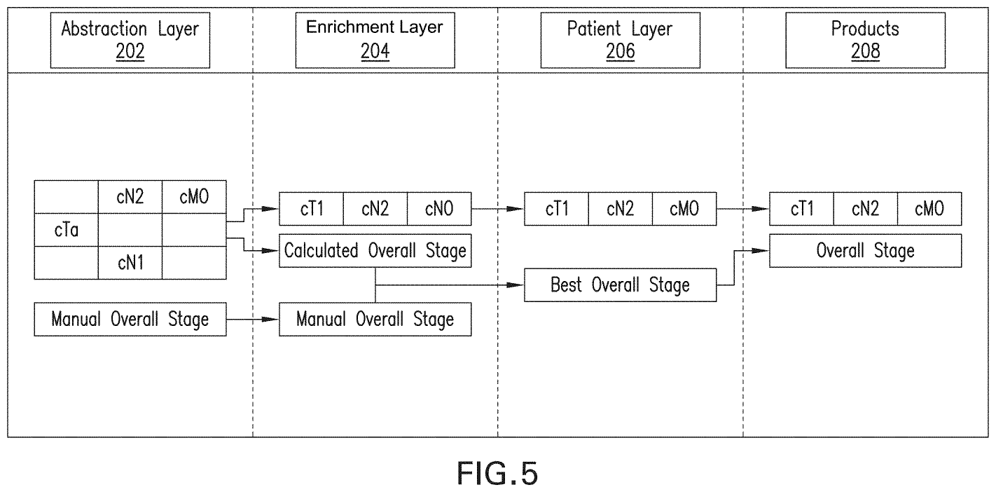

For an element that could change over time, the EL module 120 is configured to determine at least one best fact from the candidate facts that correspond to the element for a progression period corresponding to a diagnosis or progression milestone. Determination of the best fact for each element or for each element for a diagnosis or progression milestone will be described in further detail with respect to FIGS. 4-5.

In some embodiments, for all or at least some of the elements, the identified at least one best fact is presented as a suggested at least one best fact to a user via a user interface (e.g., a graphical user interface) for acceptance or verification as described above and below.

In some embodiments, for at least some elements, where the EL module identifies more than one best fact for an element that would not be treated as changing over time or more than one best fact for an element for a diagnosis or progression milestone for an element that could change over time, the EL module may send the identified more than one best fact to a conflict resolution system or module. The conflict resolution module returns an identification of a single best fact from the more than one best fact. In some embodiments, the conflict resolution system or module may present the more than one best fact to a human and receive input including a selection for the single best fact.

In some embodiments, the EL module can identify inconsistencies in the data or potential issues, such as, for cancer, a patient having more than one primary type of cancer at the same time. In some such embodiments, the EL may escalate the patient data for review by a user.

In some embodiments, the EL layer also performs calculations and derivations to obtain additional information corresponding to concepts such as age or calculated stage for the patient (204b).

In some embodiments, for at least one element, after deduplication, all of the candidate facts after deduplication are identified as best facts. For example, in some embodiments, for co-morbidities, all of the candidate facts are identified as best facts.

Once the EL module 120 has identified the best fact or best facts for each of one or more elements associated with the patient, which may be accepted or verified best facts, each of the identified best facts can be transmitted to the patient layer 206. In some embodiments, in the patient layer 206, the best version of the patient's data organized by diagnosis, patient demographics, history, and/or outcomes/treatments can be generated (206a). In some embodiments, multiple schemas are exposed to assist with data visibility and to help perform analysis on the data in the patient layer. For example, in some embodiments, EL output is stored directly in an EL schema, and that is then used to reconstruct the full patient and stored in tables across both a patient diagnosis (PDX) schema and a real-world evidence (RWE) schema, and an analytic schema is built on top of these with additional calculations and derivations. The PDX schema holds data specific to the patient's diagnosed disease, while the RWE schema holds data specific to the overall patient and is therefore disease agnostic. Alternatively, or in addition, nodal addresses and post processing to represent the patient's points of progression and progression periods can be generated (206b). In some embodiments, the data generated in the patient layer 206 can be transmitted to the products layer 208. The products layer 208 can include the application tables 127a-n. The application tables 127a-n can receive the data generated in the patient layer 206 for further use. In some embodiments, the application tables are designed to power applications with a single source rather than having to reference multiple tables and schemas. This also limits the source tables to only the data required by the application.

FIG. 3 illustrates an exemplary reduction of candidate facts to identify the best facts in accordance with an exemplary embodiment. As described above, the abstraction application 123 can access, receive, and/or retrieve data associated with a patient in the abstraction layer 202. As a non-limiting example, the abstraction module 123 can access, receive, and/or retrieve a patient's name from multiple different data repositories 170a-n. Accordingly, the abstraction module 123 can include multiple different instances of the patient's name. The abstraction module 123 can identify each instance of the data indicating the first, middle, or last name. For example, the abstraction module 123 can identify an instance of John as the first name and Doe as the last name. The abstraction module 123 can further identify an instance of John as the first name, A as a middle initial and Doe as the last name. The abstraction module 123 can further identify an instance of Jon as a first name and Doe as a last name. Each of these instances can be embodied as candidate facts corresponding to a name element of a patient. The candidate facts can be transmitted to the EL 204.

The EL 204 can receive the candidate facts, and reduce the candidate facts 204 to identify the best fact or best facts corresponding to the element using reduction rules. Each element can be associated with one or more reduction rules. A priority can be assigned to each reduction rule for each element. For example, if the EL module 120 is unable to determine a best fact from the candidate of facts for an element using a reduction rule, the next reduction rule is applied. As an example, the reduction rules can also include a rule to keep equals, keep max, and/or discard non-max. For example, if the element for patient name is designed to capture first name, middle name, and last name, the patient name that includes a first, middle, and last name is considered a better fact as compared to patient names with only first and last names. Continuing with the earlier example, the most complete data set of the candidate facts is the instance of John A. Doe. Accordingly, the EL module 120 can identify John A. Doe as the best fact corresponding to the specific element of patient name. In this example, the “max” is defined by ordering of names and “ordering” is partially defined by the presence or absence of a middle name. The “equals” here is implied, given that the goal of this process is to reduce “ties.” If a best fact is not identified as in the case of two instances of “John Doe,” both values are kept and relayed, as a list of “tied” elements, to the next reduction rule. As another example, the reduction rules can include a rule to Keep min, discard max, keep equals. In some cases, a value that is lesser is more critical to convey than greater values. In this event the EL module 120 is instructed to prefer minimum values, retain equal values, and discard higher values. As another example, the reduction rules can include if equal discard one of them. In this case, if there are two instances of the same candidate fact, the EL module 120 is instructed to discard one of the candidate facts.

As another example, the reduction rules can include a rule to keep a most frequently occurring concept by the natural ordering. In this case, some elements have an inherent priority relative to each other. Unlike an alphabetical order or a numerical order, the ordering/priority of these groups are medical in nature. For example, some histologies are more aggressive than others and would have a higher priority than others, however, this cannot be intuited simply by looking at the values corresponding to the histologies, but instead requires a specific medical ordering rule. Another example of a specific medical ordering rule is an ordering rule for menopausal status: post-menopausal has a higher priority than perimenopausal, which has a higher priority than pre-menopausal. Yet another example of a specific medical ordering rule is a rule for molecular marker testing methods: NGS has a higher priority than PCR, which has a higher priority than FISH, which has a higher priority than cytogenetics, which has a higher priority than IHC, which has a higher priority than unspecified.

For some elements, the reduction rules can include a rule that if two candidate facts are identical on some predefined number of their fields, then these two facts are candidates for reduction.

Some elements and reduction rules are specific to certain types of cancer. For example, the EL module can identify best facts for a patient with breast cancer based on information such as molecular markers estrogen receptor positive (ER+), progesterone positive (PR+), and human epidermal growth factor receptor 2 positive (HER2+). In some embodiments, if the patient has multiple different primary medical conditions or diseases (e.g., multiple different types of cancer) the EL module 120 may transmit information associated with the patient for escalation for review by a trained user to determine how to categorize the patient with respect to the primary medical condition or disease, or whether the patient can be categorized by the system.

As described above and below, in some embodiments, for at least some of the elements, the at least one best fact is presented as a suggested at least one best fact to a user via a graphical user interface, and input is received accepting or verifying the at least one best fact, or selecting a different at least one best fact from the candidate facts for the element via a process identified herein as enrichment.

The identified at least one best fact, which in some embodiments would be an accepted or verified at least one best fact for the specific element, can be transmitted to the patient layer 206. The patient layer can generate data outputs necessary to be transmitted to the products layer 208.

For each element that can change over time, the EL module 120 can associate each candidate fact corresponding to the element with a diagnosis or progression period of patient's illness or medical condition. For each element that can change over time, the EL module 120 can identify at least one best fact for each progression period having an associated candidate fact for that element. In the event that the element has only one corresponding candidate fact associated with the progression period, the EL module 120 can identify the corresponding candidate fact as the best fact corresponding to the element for the milestone. In the event that the element has more than one corresponding candidate fact associated with the milestone, the EL module 120 can identify at least one best fact corresponding to the element for the progression period from the more than one candidate facts based on reduction rules specific to the element.

For at least some elements, the EL module 120 can derive a best fact for an element associated with the patient based on one or more of the other candidate facts extracted from the data and one or more medical rules. In the event the derived candidate fact corresponds to an element that is unchanging over time, the EL module 120 can identify the best fact based on reduction rules specific to the element by comparing the derived candidate fact to one or more candidate facts extracted from the data for the element. In the event that the element has more than one corresponding candidate fact associated with the milestone, the EL module 120 can identify the best fact corresponding to the element for the milestone from the more than one corresponding fact based on reduction rules specific to the element comprising comparing the derived candidate fact with one or more candidate facts extracted from the data.

For each element that can change over time, the associating of each candidate fact corresponding to the element with a progression period associated with diagnosis or progression milestone can be based on time windowing, meaning events that happen within a given time window are assigned to the time window, which may be a time window with respect to an initial diagnosis window or a progression track window. The initial diagnosis window, which is the initial progression period, is defined by the time between the date of initial diagnosis and the first time that the patient progresses. Subsequent “progression track” time windows, also referred to herein as progression periods, are defined by a start of the progression date up until one of (1) a subsequent progression date; (2) patient death; or (3) “today”, meaning the patient is still alive and has not progressed again, effectively an “undefined” end date.

FIG. 4 illustrates the application of medically based rules for identification of a best fact in accordance with an embodiment. In one embodiment, the EL module 120 can calculate or derive the best fact or best facts corresponding to an element associated with a patient which changes over time or which is unchanged over time. As an example, candidate facts 402-408 correspond to a patient's overall stage at a diagnosis or a progression milestone of patient's illness or medical condition. Based on reduction rules specific to the element “overall stage” that prefer pathologically determined stage to clinical stage, the EL module 120 can determine that candidate facts 404 and 406 provide the more accurate information with respect to the patient's overall stage than do candidate facts 402 and 408, which are not pathologically determined, based on the data provided in candidate facts 402-408 indicating how the determination of the stage was made.

Unlike most other elements, all comorbidity facts are considered “best” so there is no requirement to select a single best fact for comorbidity. The reduction rules specific to comorbidity reflect this.

In some embodiments, the EL module 120 determining a best fact for an element includes determining that one or more candidate facts are inaccurate or incomplete based on other candidate facts using calculations or determinations.

In some embodiments, the candidate facts include both candidate facts from the patient record and calculated or derived candidate facts. FIG. 5 illustrates an example using provided candidate facts from the patient record and calculated and/or derived candidate facts in accordance with an exemplary embodiment. At the abstraction layer 202 the abstraction module 123 can abstract patient data resulting in candidate facts corresponding to an element associated with a progression period corresponding to a diagnosis or progression milestone of the patient's illness or medical condition. At the EL layer 204, the EL module 120 can calculate the best fact from the candidate facts using the data in the candidate facts and reduction rules. The calculated best fact is promoted to the “best overall stage”. The “best overall stage” can represent the most accurate diagnoses or progression milestone of a patient's illness or medical condition.

FIG. 5 schematically illustrates the aggregation or abstracted data, and the “best” information being selected for use in eventual downstream products. The example illustrates how overall stage can be explicitly stated by the physician and abstracted as such, while TNM, which make up the components of the overall stage calculation, is also abstracted. All of the information is stored in the database, and then EL logic is used to determine which version (calculated stage from TNM facts or overall stage explicitly stated by the physician) best represents the patient for use in the nodal address and otherwise. In some embodiments, the identified best overall stage as determined by the EL 204, the calculated overall stage, and the manual overall stage are presented to a user via a graphical user interface for acceptance or verification of the best overall stage before information regarding the best overall stage is output to the patient layer 206.

The EL module is configured to perform clinical validation, meaning given the data set, the most likely accurate data for a particular fact or value is data point X, which includes implementing medically-based reduction rules. For example, the rule described above associated with overall stage that indicates a preference for stage determined from a pathology report as opposed to clinically determined is a medically-based reduction rule.

In some embodiments, the system may determine whether the output best facts and/or the patient are suitable for various downstream applications. For example, in some embodiments, at the patient layer 206, the EL module 120 may use the calculated “best overall stage” to determine whether the patient data should be sent on to other applications such as RWA. In some embodiments, a patient or the patient's output best fact data can be deemed unqualified or unsuitable to be provided to downstream applications based on total elements identified, clinically significant conflicts in the data, or other factors. The EL therefore acts as a fact gateway where abstracted facts are reduced, derived and calculated, which prevents erroneous or incomplete patient data from progressing, before they are promoted downstream.

In some embodiments, for at least some elements, an identified best fact for the element for the progression period is not displayed to a user for acceptance and verification unless there is more than one identified best fact for that element for the progression period and user input is received that selects one best fact from among the more than one best fact. In such an embodiment, the system displays the more than one best fact for user selection when the reduction rules fail to identify a single best fact. This may be described as conflict resolution. In some embodiments, the conflict resolution may include sending an inquiry to a health care provider or health record provider for additional information or confirmation to determine the best fact for the element.

In some embodiments, for at least some elements, an identified best fact for the element is always displayed as a suggested best fact to a user for acceptance and verification, whether there is only one identified best fact or whether there is more than one identified best fact for the element for the progression period. In some such embodiments, at least some of the other candidate facts are displayed as well as the suggested at least one best fact, which is identified as a suggested best fact.

A workflow in the EL module describes a logical sequence of operations that are carried out in order to obtain a predefined result, e.g., an order of implementation of rules for an element. In some embodiments, the system includes or employs a rules engine that is a module, which may be implemented as a software component that enables non-programmers to add or change rules or workflows in the EL module.

The term “decision table” as used herein refers to a list of decisions and their criteria. Designed as a matrix, it lists criteria (inputs) and the results (outputs) of all possible combinations of the criteria. It can be placed into a program to direct its processing. The program is changed by changing the decision table. In some embodiments, decision tables are employed to enable a non-programmer to add or change rules or workflows in the EL module.

In some embodiments, at least some of the reduction rules employ fuzzy logic. The term “fuzzy logic” as used herein refers to a type of logic for processing imprecise or variable data, which, in place of the traditional binary values, employs a range of values for greater flexibility.

As noted above, in the EL 204, the EL module 120 identifies the best facts for each element from the candidate facts received from the abstraction module 123 in the abstraction layer 202, and the EL module 120 generates one or more outputs. The outputs include progression output, a time series output, or both. The time series output presents the best facts corresponding to the elements associated with the patient over time, as a series of events. The progression output presents the best facts corresponding to the elements associated with the patient in view of progression periods corresponding to progression milestones of the patient's illness and/or medical condition. The time series output and the progression output can be used by applications (e.g., real world data (RWD), real world evidence (RWE)) in the product layer 208.

In some embodiments, RWD receives a time series output. In RWD, facts are presented as a time series corresponding to a set of tables. The tables can be delivered to customers in a data file or a series of data files. In some embodiments, the data files can be CSV/XLS files.

In some embodiments, RWE receives progression output. In RWE, facts are used to build progression tables for use in a number of other products and solutions. Progressions provide analytically-relevant windows over the patient journey and benchmark the frequency and distribution of treatment changes.

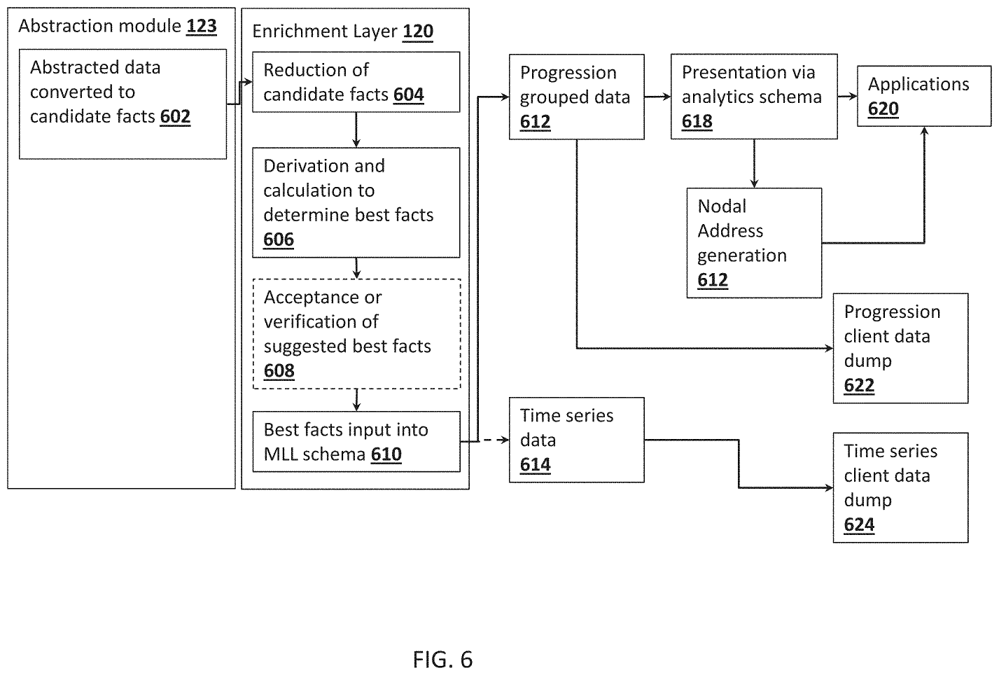

FIG. 6 illustrates an EL workflow in accordance with an exemplary embodiment. As described above, EL data can be output as progression data or time series data or both. In one embodiment, the abstraction module 123 can abstract data associated with a patient to convert the data into candidate facts 602 corresponding to elements associated with a patient. The candidate facts are transmitted to or accessed by the EL module 120. The EL module 120 executes a reduction process 604 to reduce the candidate facts corresponding to each element based on the reduction rules. For at least some elements, the EL module 120 executes a derivation and calculation process 606 to derive and calculate best facts from the reduced candidate facts corresponding to each element. In some embodiments, for at least some of the elements, the one or more best facts are displayed to a user in a graphical user interface for acceptance or verification 608. This step is outlined with a dotted line as it may not be included in all embodiments. The best facts, which may be accepted or verified best facts, are input into an EL schema 610. The EL schema is the set of tables that store the EL output. Each medical concept abstracted is processed through the EL and output to storage in its own table. For example, in some embodiments, all “lymphovascular invasion” facts abstracted in the platform would be processed through the same EL rules and output together into the same storage table with their attributed patient_id and progression_track_id for reference.

In some embodiments, the EL module 120 can transmit the best facts from the EL schema 610 for the patient as progression grouped data 612 and/or a time series data 614. In some embodiments, the EL outputs all the abstracted data for elements, not just the best facts in two forms: one for time series data that reflects all data abstracted for a patient based on the timeline of events following the date of diagnosis, and one for progression based data that groups the time series data based on their inclusion within the defined “progression windows.” The best facts in the stored progression grouped data corresponding to the each element 612 are presented via an analytics schema 618 for use in applications such as RWA. In some embodiments, the best facts are also stored as time series data.

In some embodiments, the best facts are used to generate a nodal address 612. In some embodiments, the nodal address is a refined nodal address (defined below). In some embodiments, the nodal address is a provisional nodal address where attributes are assigned for at least a minimum subset of a set of treatment relevant variables. In some embodiments, the nodal addresses are generated based on data from the analytics schema. In some embodiments, the nodal addresses are based on data from the EL schema. In some embodiments, the generated nodal address may be used as input for one or more applications.

By centralizing the logic or rules employed for reduction and determination of the best facts, into the EL, the EL module ensures that each downstream application is using the same concept of progressions for each patient at all times. This is critical to ensuring that when a clinical record of one of the patients is referenced in any product or application that uses output from the EL, the data used is exactly the same across all products and applications, with the only difference being in the way that patient's data is conveyed. The best facts from the progression grouped data 612 can be transmitted or provided in one or more progression client data dumps 622.

The best facts in the time series data 614 can be used to generate time series client data dumps 624 in some embodiments. This data can be utilized for increased scrutiny of the patient timeline and events. For instance, while a downstream user that is a health care provider may only want to see summary information about their own patients, or macro information across many patients, a downstream user in the pharmaceutical industry may want to see each and every instance that a lab value was measured. These disparate needs by downstream users require a different approach to assembling the patient story.

In some embodiments, based on derived and explicit concepts, information associated with a patient's cancer can be presented as a timeline of events from diagnosis to the present or to expiration for a deceased patient.

The most critical difference between the progression grouped data and the time series data is the presence and restriction of time windows to progression periods, which may also be described as Progression Tracks herein. Rather than being bound to the time windows of Progression Tracks, the Time Series data is simply represented as a function of time. The result is that the data can be more easily stratified outside by external applications from different providers, but the conclusions drawn from it can vary by each interpreter.

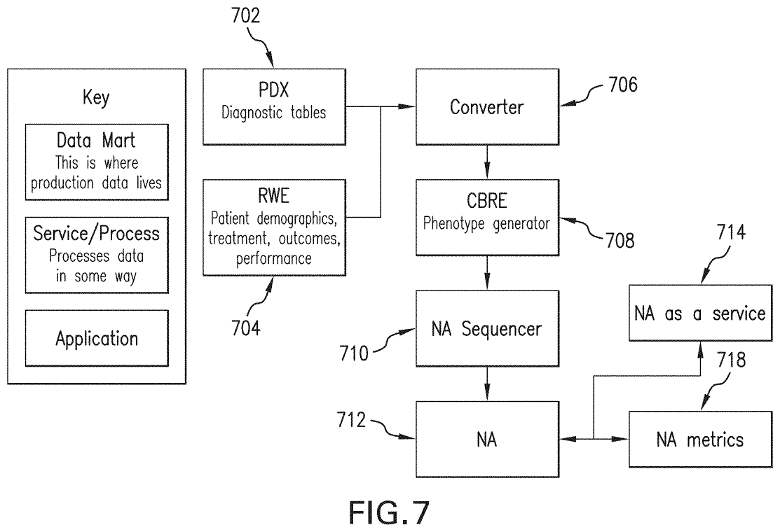

FIG. 7 illustrates generation of a nodal address from the EL output in accordance with an exemplary embodiment. Details regarding the generation of nodal addresses, which may be referred to as COTA nodal addresses or CNAs, may be found in U.S. Published Patent Application No. 2015/0100341, which is incorporated by reference herein in its entirety. Details regarding the generation of nodal addresses that are provisional nodal addresses or refined nodal addresses may be found in U.S. Provisional Patent Application No. 62/900,135, which was filed on Aug. 13, 2019, by the Applicant of the present application, and in U.S. Patent Application Publication No. US 2021/0082573, each of which is incorporated by reference herein in its entirety.

A nodal address can be assigned to a set of personal health information regarding a patient based on values, referred to as attributes, of the preselected variables included in the nodal address (e.g., in the provisional nodal address or in the refined nodal address). Preselected variables can include treatment relevant variables, and can also include prognosis or outcome relevant variables that may or may not be relevant to treatment.

Nodal addresses, which may be provisional nodal addresses, facilitate early treatment decisions for a patient. For example, in some embodiments, the nodal address to which a patient is assigned is associated with a bundle of predetermined patient care services, and information regarding the predetermined patient care services may be provided to a healthcare provider of the patient or a healthcare payer of the patient. Further, as additional or updated information relevant to treatment of the patient is received, the nodal address is updated or changed as needed based on the additional or updated information relevant to treatment. If a different bundle of predetermined patient care services is associated with the updated or changed provisional nodal address, information regarding the different bundle of predetermined patient care services is provided to a healthcare provider of the patient or to a payer for healthcare of the patient.

In some embodiments, initial data regarding a patient will be provided to the system or for the method at or shortly after diagnosis of the patient. At the time at which a provisional nodal address or an updated nodal address is assigned, the patient data provided may include sufficient information to determine a recommended course of treatment for the patient, but insufficient information to provide a prognosis-related expected outcome with respect to occurrence of a defined end point event (e.g., overall survival, progression free survival, or disease free survival) for the patient. Instead of waiting to receive information relevant to the prognosis-related expected outcome, but not relevant to treatment, before assigning the patient to a nodal address, assigning the patient to a provisional nodal address that only incorporates treatment relevant information enables the system or method to assist health care providers or health care payers in guiding treatment decisions for the patient information, especially early in the disease process after diagnosis.

In some embodiments, the nodal address associated with the patient for a progression period is used to determine a prognosis related expected outcome for the patient. This is explained in further detail in U.S. Published Patent Application No. 2015/0100341, U.S. Provisional Patent Application No. 62/900,135, filed on Aug. 13, 2019, and U.S. Patent Application Publication No. US 2021/0082573, each of which is incorporated by reference herein in its entirety. In some embodiments, after additional information regarding the patient is received that includes at least a minimum amount of information relevant to a prognosis-related expected outcome, the patient is assigned to a refined nodal address that is used to determine a prognosis related expected outcome for the patient. In some embodiments, the refined nodal address is used for risk adjustment of expected outcome for the patient. The EL output can include best facts corresponding to elements associated with a patient. The best facts can represent the most accurate information associated with the patient and the patient's illness and/or medical condition. Accordingly, the EL output can be used to generate a nodal address for a patient corresponding to a diagnosis or progression period corresponding to a diagnosis or progression milestone using the best facts as attributes.

In some embodiments, an EL output can be expressed in a patient diagnosis PDX 702 schema specific to the patient's diagnosed disease and/or in a RWE schema 704 that is disease agnostic and holds data specific to the overall patient (e.g., i.e., patient demographics treatments, outcomes, and performances). In some embodiments, the EL output, which may be in the form of data from the patient diagnosis PDX schema 702 and data from the RWE schema 704, can be transmitted to a converter 704 that can convert the PDX or RWE data format data into phenotype Clinically-Based Rules Engine (CBRE)-compatible data to be transmitted to a phenotype generator CBRE 708. The term “phenotype” as used herein means any observable characteristic of a disease without any implication of a mechanism. In some embodiments, the converter 706 is unnecessary and the output from the EL can be handled as input into the phenotype generator CBRE 808 without conversion. The phenotype CBRE 708 can generate a phenotype based on the input data. The phenotype generator CBRE 708 can transmit the data regarding the generated phenotype to the nodal address sequencer 710. The nodal address sequencer 710 can determine if the generated phenotype is a new phenotype of the disease that did not have a previously generated nodal address. If so, it can generate a new nodal address. If not, a previously generated nodal address will be assigned to the patient. If there is not enough information to generate a nodal address, the nodal address sequencer 710 may not generate the nodal address. In some embodiments, if no nodal address is generated, a message may be transmitted indicating that there was insufficient information to generate a nodal address. The nodal address module 712 can generate a nodal address for patients and progressions based on the cancer. In some embodiments, the nodal address may be sent to a user, a client, or an application. For example, in some embodiments, the nodal address is sent to a nodal address metrics application 718 and/or a nodal address as a service 714 application.

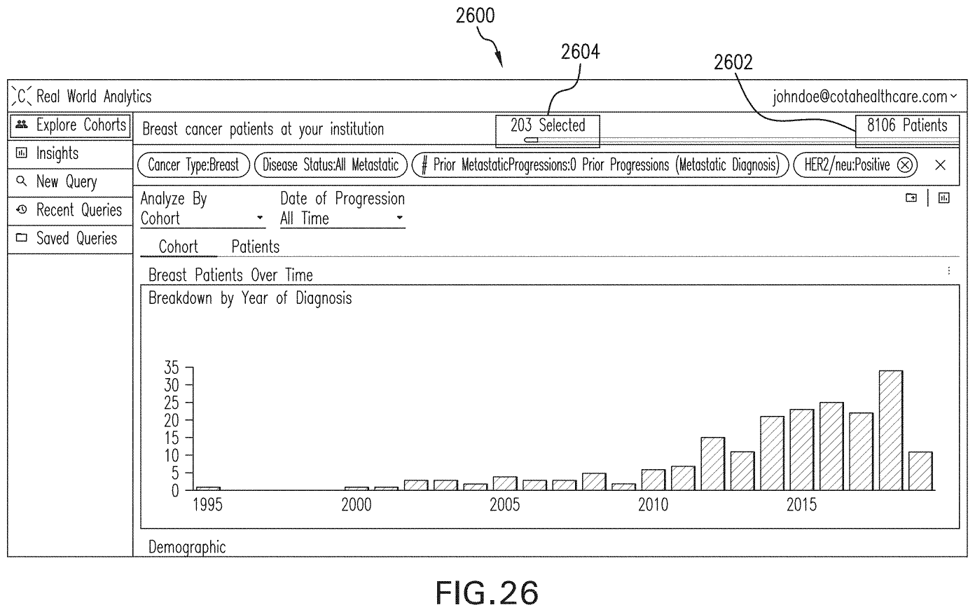

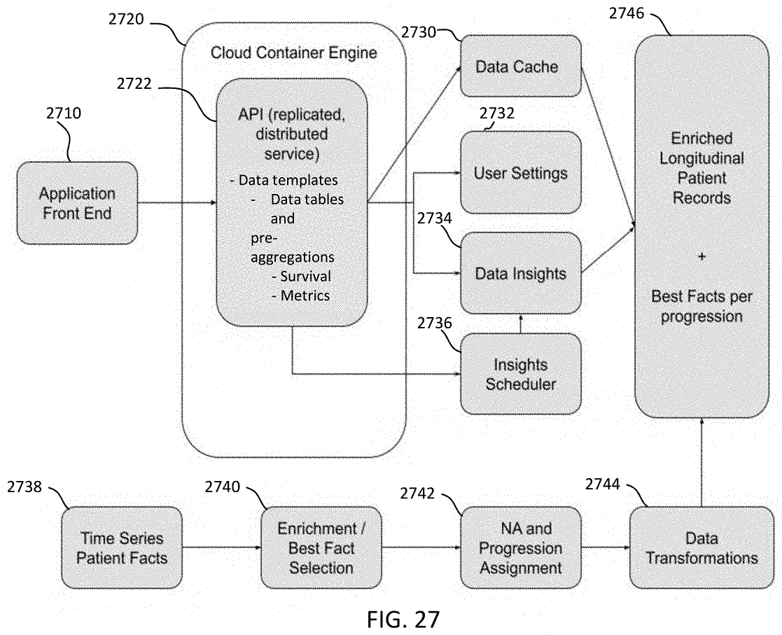

In some embodiments, a nodal address based on best facts and or best facts themselves may be employed for analysis of patient outcomes, for analysis of patient treatment, for identification of a patient as a candidate for a specific treatment, for analysis of outliers in treatment or outcome, for reduction of variance in treatment, or for identification of treatment plans or options appropriate for a patient. Such uses of a nodal address or other patient information and systems and methods that employ nodal addresses or other patient information that may be refined by best fact enrichment are described in U.S. Pat. Nos. 9,378,531; 9,646,135; and 9,734,291, each of which is incorporated herein by reference in its entirety. Additional description of systems and methods incorporating analytics employing nodal addresses based on best facts and/or best facts themselves for analysis of patient outcomes, for analysis of patient treatment, for identification of a patient as a candidate for a specific treatment, for determining a treatment plan appropriate for a specific patient's disease, for analysis of outliers in treatment or outcome, for reduction of variance in treatment, and/or for identification of treatment plans or options appropriate for a patient is described below in connection with FIGS. 26 and 27.

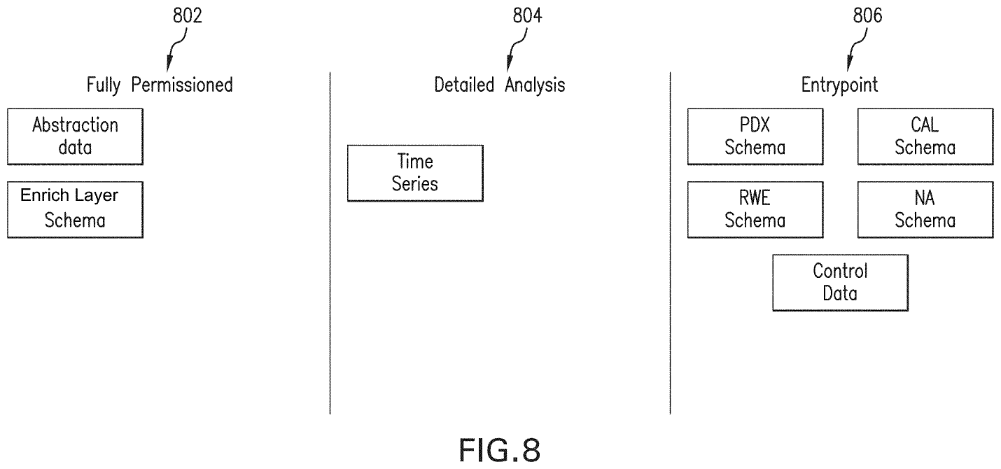

FIG. 8 illustrates a read-only database permission model in accordance with an exemplary embodiment. The read-only database permission model can be used by the EL module 120 and can divide data into three output levels, fully permissioned 802, detailed analysis 804, and entrypoint 806.

The fully permissioned output level 802, includes the most raw data collected from the abstraction effort, and the lightest processing of data in the EL. This output data is the most complex analytically, but is the most difficult to parse. The detailed analysis output level 804 includes time series data that allows for detailed analysis and is particularly suited to the life sciences vertical (e.g., a business unit that integrates across multiple segments of the life sciences industry such as medical informatics, business intelligence, through discovery for biotechnology companies, pharmaceuticals, medical devices, etc.) or analysis by pharmaceutical industry end users. This is a decoupled view of the progression-based model and encourages more advanced analysis based on the time-series nature of the data. The entrypoint output level 806 can include the analysis that has already been performed on this data and is the result of all facets of the EL, and is designed for those who need to generally consume patient data in more rich, predefined structures. Multiple schemas can be exposed to assist with data visibility and to help perform analysis on the data in the patient layer.

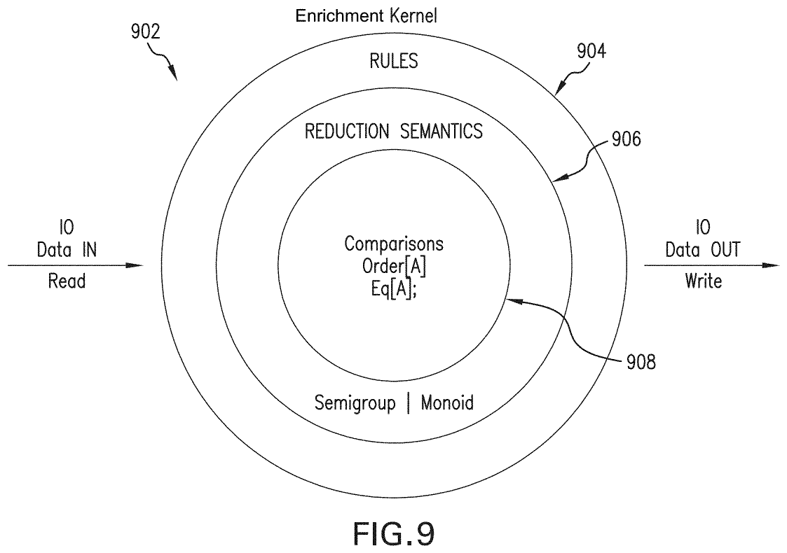

In an operating system, a kernel is a computer program that manages input/output requests from software, and translates them into data processing instructions for the central processing unit and other electronic components of the computer. FIG. 9 illustrates an exemplary EL kernel 902 implemented in the EL module, in accordance with an exemplary embodiment. The data flow into the EL kernel 902 can include candidate facts generated from the abstraction module 123, a subset of the fact metadata, and control data. This design gives EL the flexibility to use all three sources when applying medical and reduction rules against the data contained within candidate facts.

As an example, in some embodiments, EL kernel 902 can receive molecular markers, histologies, and/or oncotrees, International Statistical Classification of Diseases and Related Health Problems 10th Revision (ICD 10) codes as control data. For example, in some embodiments the control data includes all ICD10 codes that represent cancer. In some embodiments, the control data also includes all ICD9 codes that represent cancer. In some embodiments, the control data can include anything used to describe the explicit set of values proposed for capture for any mapped data. ICD codes are alphanumeric codes used by doctors, health insurance companies, and public health agencies across the world to represent diagnoses. Each code describes a particular diagnosis in detail. The first 3 characters define the category of the disease, disorder, infection or symptom. For example, codes starting with M00-M99 are for diseases of the musculoskeletal system and connective tissue (like rheumatoid arthritis), while codes starting with J00-J99 are for diseases of the respiratory system. Characters in positions 4-6 define the body site, severity of the problem, cause of the injury or disease, and other clinical details. In the rheumatoid arthritis example above, the fifth character defines the body site and the sixth character defines whether it's the left or right side. A three in the fifth character position denotes it's a wrist that's affected. A two in the sixth character position denotes it's the left side of the body that's affected. Character 7 is an extension character used for varied purposes such as defining whether this is the initial encounter for this problem, a subsequent encounter, or sequela arising as a result of another condition. The EL module 120 can pull in ICD 10 code information or data as needed.

Oncotree groups represent differences between histologies which should be treated differently or not in terms of treatment. For example, some differences in histology may not require different treatment. Oncotrees map different histologies into groups that can be treated similarly. The EL module 120 can employ a blended histology concept based on oncotree groups.

The same histology may be used for different types of cancer (e.g., adenocarcinoma for lung, breast, or colon cancer). Determining what oncotree group corresponds to a particular histology requires referencing an ICD code to determine the cancer subtype for the histology.

For a cancer specific implementation, most of the control data is agnostic as to type of cancer. In some embodiments, the system receives a selection of some values for control data through a graphical user interface (e.g., through drop down menus).

Some of the control data specifies which values are allowable for some facts. For example, the control data can include, ER+, ER amplified and ER- as allowable data for an estrogen receptor (ER). In some embodiments, some control data is selected or specified by an operator of a system via a graphical user interface (e.g., via drop-down menus, which may include multi-level drop down menus).

In the rules layer 904 of the EL kernel 1002, medical rules can dictate reduction steps, including deduplication, ordering, and comparisons. In some embodiments, duplication results, at least in part, from facts coming from multiple different sources, necessitating deduplication. Elements that have a clear medical hierarchy or a decision tree for selecting one item over another can be described in the rules layer 904.

In the reduction semantics layer 906 of the EL kernel 902, reduction semantics provide the “translation layer” between medical rules and abstract algebra, effectively converting medical rules into algebraic concepts. These algebraic concepts further stratify the medical rules into mathematical rules, allowing EL to execute reduction logic programmatically.

In the comparisons layer 908 of the EL kernel 902, the output from the reduction semantics layer is a list per element. The comparisons layer 1008 orders and ranks the results within and across these lists as required. The comparison layer 1008 is where (one or more) “winners” are decided.

Depending on the scope of the medical concept, any layer might write “Data Out.” This is dependent on the order of operations as determined within the set of rules. Data Out is output that is written to a table within the EL schema.

As a non-limiting example, the reduction of candidate facts can be represented by numbers. For an example set of natural numbers (0, 1, 2, 2), using a reduction rule the reduction will result in (2, 2). This is because 0 and 1 are less than 2 and are thus discarded. As both of the 2 s are the max value in this set of numbers, both of them are retained. Applying another reduction rule can reduce (2,2) into a single value. In short: (0, 1, 2, 2,)→(2, 2)→2.

In some embodiments, the EL module processes input data and candidate facts in the form of Shapes. By design, similar arithmetic operations can be applied to Shapes, which enables them to be compared against specific criteria. The EL module can reduce a sequence of Shapes in the form of candidate facts into one or more other Shapes. To reduce a sequence of Shapes, all that is required is the ability to reduce two shapes, which is referred to as “combining” the two shapes, and then apply the same reduction semantics across the sequence of Shapes. In some embodiments, the EL module uses a custom operator to “combine” Shapes. The Shapes and the custom “combine” operator have associativity, meaning that it does not matter what order in which the series of Shape are combined. This enables the EL to combine different pairs of shapes in a sequence independently on multiple processors or computing devices and combine or merge the results later, enabling faster and more efficient processing. If two Shapes are “combined” the result is always a Shape.

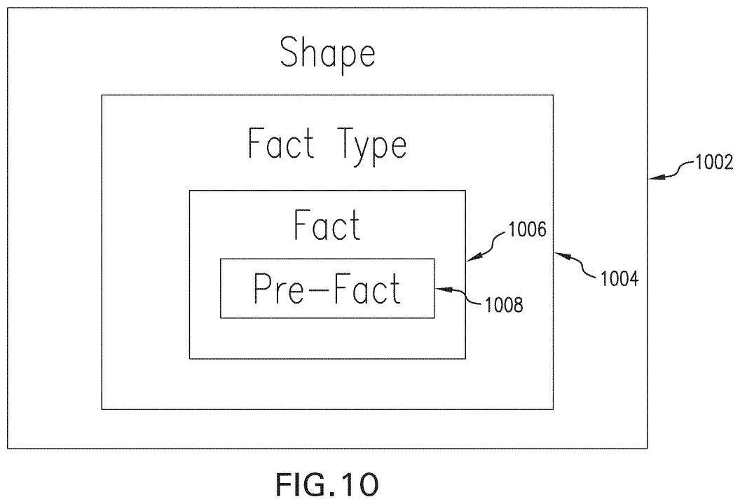

FIG. 10 illustrates a shape structure that can be employed in representing a candidate fact in accordance with some exemplary embodiments. A shape 1002 can represent a medical concept with attributes, the control data of the attributes, and what is required/optional within the scope of the concept. The shape 1002 can be an element associated with a patient. A shape 1002 can be a template, and defines how any particular patient data “input form” works. A fact type 1004 can be a class or specific indicator of the medical concept, such as “Estrogen,” “Receptor,” “Name,” or “Eastern Cooperative Oncology Group (ECOG)” scale of performance status (which describes a patient's level of functioning in terms of their ability to care for themself, daily activity, and physical ability (walking, working, etc. to name a few). A fact type can be a “child” of a shape 1002. In the case of an ECOG, for example, its unique shape 1002 also makes it a unique fact type 1004. A fact 1006 is the actual saved instance of a fact type 1004, such as “Abstractor A saved an ECOG from patient document XYZ on Monday at 3:00 PM.” All facts 1006 are associated with their fact type 1004 (and, correspondingly, a Shape), a timestamp, a user, and the document from which the fact 1006 was generated. A pre-fact 1008 is incomplete relative to the required input fields of a Shape. For example, a tumor registry can be received that has a column for overall stage, but the date on which the overall stage was identified is missing.

As an example, ECOG performance status is supposed to be collected every time a patient diagnosed with cancer visits a hospital. It may be important for a hospital end user to determine that the hospital measured ECOG at every visit. Each ECOG collected can be a candidate fact corresponding to an element associated with a patient. The EL module 120 can identify if within the same visit ECOG was measured n times with the same result. The EL module 120 can de-duplicate and consider it as one result. If the ECOG results are different, the EL module 120 may identify the best fact as the highest one or it might get escalated to a separate conflict resolution module or to the QA team if a conflict has been detected for resolution of the conflict, based on the reduction rules.

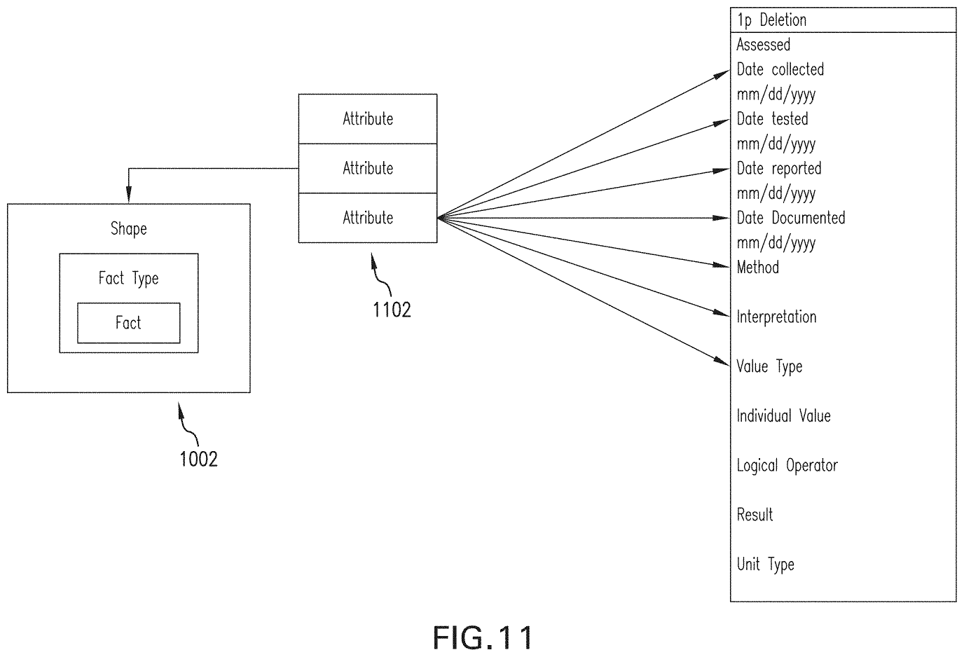

FIG. 11 illustrates attributes associated with a shape in accordance with an exemplary embodiment. An attribute 1102 can be a single “input field” represented within a shape 1002. Attributes 1102 may have control data such as a drug list or methods, and may allow free text, or may require numeric-only patterns.

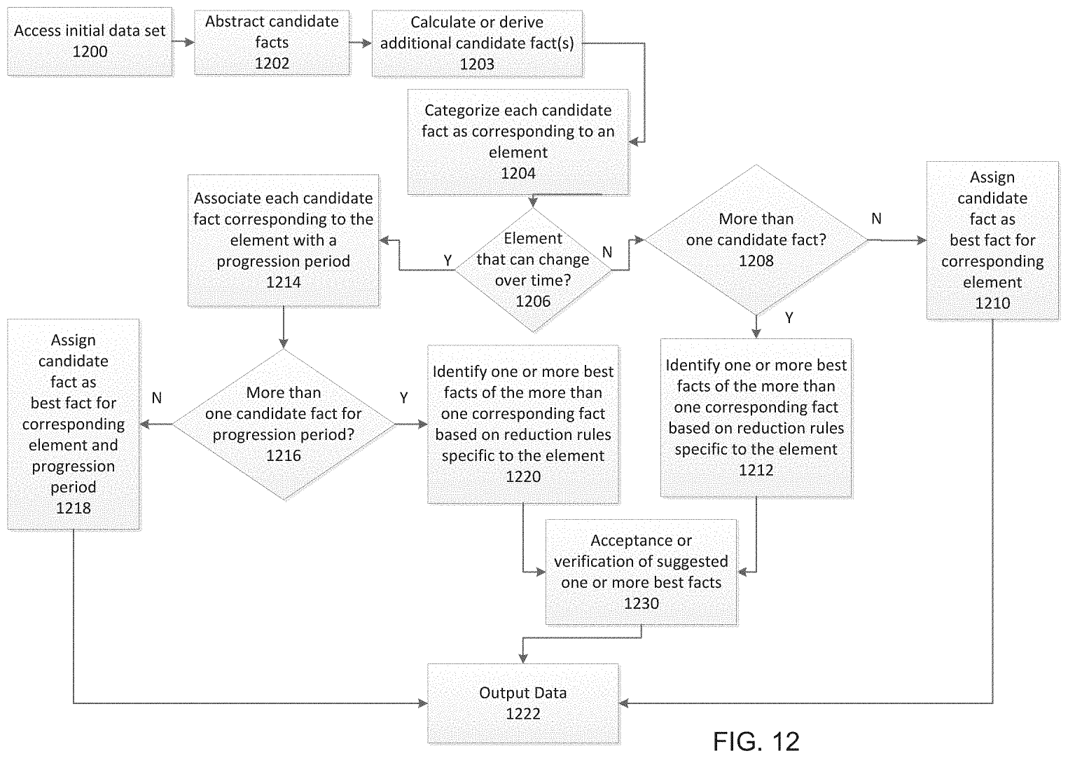

FIG. 12 is a flowchart illustrating the process of identifying one or more best facts for a corresponding element in accordance with some embodiments. In operation 1200, the abstraction module 123 accesses or receives an initial set of data records associated with a patient. The initial set of data records can include information regarding a patient, the patient's illness, and/or the patient's treatment. In operation 1202, the abstraction module 123 can abstract candidate facts from the initial set of data records. Each of the candidate facts can be represented as a data set. In some embodiments and for at least some elements, one or more additional candidate facts may be derived or calculated from the candidate facts or other data in the initial data set after abstraction of the candidate facts in operation 1203. As a non-limiting example, in some embodiments the EL module 120 derives an overall stage from TNM coding in the accessed data records. In some embodiments, the EL module can also evaluate a stage of the tumor from other information in the accessed data records. The EL module 120 can compare the evaluated stage to the derived stage as a function of TNM decide whether to escalate the patient because stages disagree, or confirm that that the stages appear consistent. In operation 1204, the abstraction module 123 can categorize each candidate fact as corresponding to an element associated with the patient. More than one candidate fact can correspond to an element. The elements can be associated with information regarding a patient's personal information or information regarding a patient's medical condition or illness. Some elements such as biological gender at birth or birth date may be expected not to change over time or with progression of an illness. Other elements such as disease stage or treatments may be expected to change over time.

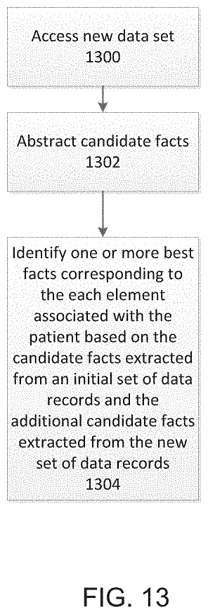

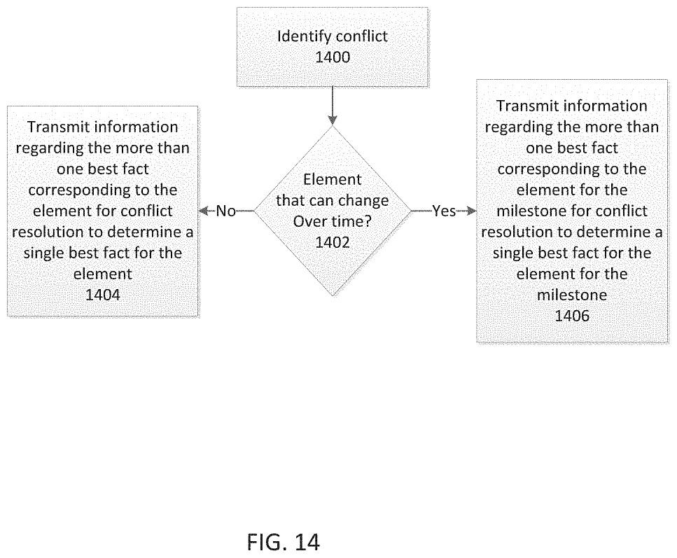

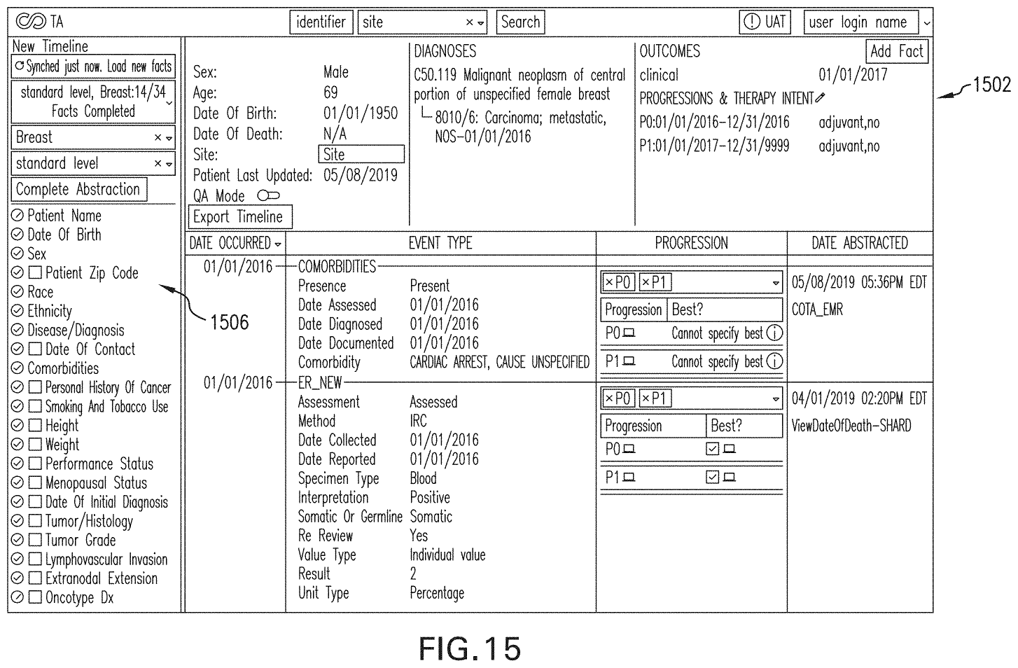

In operation 1206, the EL module 120 can determine whether the element can change over time or with disease progression. In some embodiments, the properties of a Shape with which the element is associated will indicate whether the element should be treated as unchanging element or as an element that can change over time or with disease progression. In operation 1208, for elements treated as unchanging over time the EL module 120 determines whether the element has more than one corresponding candidate fact. In operation 1208, where the element has only one corresponding candidate fact, the EL module 120 can identify the corresponding candidate fact as the best fact. In some embodiments and for at least some elements, the identified best fact may be subject to acceptance or verification by a user. In operation 1212, where the element has more than one corresponding candidate fact, the EL module 120 can identify one or more best facts of the more than one corresponding fact based on reduction rules specific to the element. In some embodiments, if more than one best fact is identified for the corresponding element in operation 1212, the system may send the more than one best fact to another module or system for determination of a single best fact for the corresponding element for use later in the process (not shown). In some embodiments and for some elements, whether or not more than one best fact is identified in operation 1212, the one or more best facts are presented as suggested best facts to a user via a user interface for acceptance or verification 1230 as described below with respect to FIGS. 13 and 14 prior to outputting data corresponding to the one or more best facts 1222. In operation 1214, for each element that can change over time, the EL module 120 can associate each candidate fact corresponding to the element with a progression period corresponding to a diagnosis or progression milestone. In operation 1216, for each element that can change over time, the EL module can determine whether the element has more than one corresponding candidate fact for the progression period. In operation 1218, where the element has only one corresponding candidate fact associated with the milestone, the EL module 120 can identify the corresponding candidate fact as the best fact corresponding to the element for the milestone. In operation 1220, where the element has more than one corresponding candidate fact associated with the milestone, the EL module 120 can identify at least one best fact corresponding to the element for the milestone from the more than one corresponding fact based on reduction rules specific to the element. In some embodiments, if more than one best fact is identified as corresponding to the element for the milestone in operation 1220, the system may send the more than one best fact to another module or system for determination of a single best fact for the corresponding element for the milestone for use later in the process (not shown). In some embodiments and for some elements, whether or not more than one best fact is identified in operation 1220, the one or more best facts are presented as suggested best facts to a user via a user interface (e.g., a graphical user interface) for acceptance or verification 1230 as described below with respect to FIG. 15 prior to outputting data corresponding to the one or more best facts 1222. In operation 1222, the EL module 120 can output data including the best facts associated with the patient.

FIG. 13 is a flowchart depicting a process of determining the best fact in response to receiving additional data in accordance with some embodiments. In operation 1300, the abstraction application 123 can access a new set of data records, including information regarding a patient, the patient's illness, and/or the patient's treatment. In operation 1302, abstraction application 123 can extract additional candidate facts corresponding to elements associated with a patient. In operation 1304, the EL module 120 can identify one or more best facts corresponding to the each element associated with the patient based on the candidate facts extracted from an initial set of data records and the additional candidate facts extracted from the new set of data records. In some embodiments, this may be done using operations described with respect to operations 1206 to 1222 in FIG. 12. In some embodiments, the EL module 120 can determine a best fact corresponding to an element associated with a patient has been identified based on the initial data set. The EL module 120 can determine whether a new best fact can be identified from the candidate facts extracted from the additional data set.

FIG. 14 is a flowchart depicting a process for conflict resolution in accordance with some embodiments. In operation 1400, the EL module 1400 can identify a conflict between more than one best fact of the candidate facts corresponding to an element, in determining the best fact for the element. In 1402, the EL module 120 determines whether the element changes over time. In 1404, where more than one best fact is identified corresponding to an element for an element that is unchanging over time, the EL module 120 can transmit information regarding the more than one best fact corresponding to the element for conflict resolution to determine a single best fact for the element. In operation 1406, where more than one best fact is identified as corresponding to an element associated with a milestone for an element that can change over time, the EL module can transmit information regarding the more than one best fact corresponding to the element for the milestone for conflict resolution to determine a single best fact for the element for the milestone.

Some embodiments employ user acceptance or verification of the best facts for at least some elements instead of or in addition to the conflict resolution shown in FIG. 14. In some embodiments, for at least some of the elements, the identified one or more best elements are subjected to acceptance or verification (see operation 1230 in FIG. 12) prior to output of data from the EL. For example, in some embodiments and for at least some of the elements that are unchanging over time, identifying the at least one best fact corresponding to the element includes presenting the at least one best fact as a suggested at least one best fact corresponding to the element to a user via a graphical user interface and receiving one or more of an acceptance of the suggested at least one best fact; an identification of at least one other candidate fact that is not a suggested best fact as at least one best fact; and a rejection of the suggested at least one best fact as a best fact. Where a rejection of the suggested at least one best fact is received, the suggested at least one best fact is no longer identified as the at least one best fact corresponding to the element. Where an acceptance of the suggested at least one best fact is received, the at least one best fact is identified as an accepted best fact. Where an identification of at least one other candidate best fact that is not a suggested best fact as the at least one best fact is received, the at least one other candidate best fact is identified as an accepted at least one best fact. In such an embodiment, for this element, the output best fact would be an output accepted best fact.

In some embodiments, for at least some of the elements that can change over time, identifying at least one best fact for each progression period having an associated candidate fact for the element further includes: presenting the at least one best fact for the progression period as a suggested at least one best fact corresponding to the element; and receiving one or more of: an acceptance of the suggested at least one best fact as at least one best fact; an identification of at least one other candidate fact that is not a suggested best fact as at least one best fact; and a rejection of the suggested at least one best fact as a best fact. Where a rejection of the suggested at least one best fact is received, the suggested at least one best fact is no longer identified as the at least one best fact corresponding to the element for the progression period. Where an acceptance of the suggested at least one best fact is received, the at least one best fact is identified as an accepted best fact for the progression period. Where an identification of at least one other candidate best fact that is not a suggested best fact as the at least one best fact is received, the at least one other candidate best fact is identified as an accepted at least one best fact for the progression period. In such an embodiment, for this element, the output best fact for a progression period would be an output accepted best fact.

FIG. 15 is a screenshot of a portion of a graphical user interface 1502 that may be employed for reviewing suggested best facts. The GUI includes identification of progression time periods and a listing of elements 1506 for which candidate facts and suggested best facts can be displayed. In some embodiments, the graphical user interface may be associated with the abstraction platform. In some embodiments, the graphical user interface enables a user to accept, verify, or identify progressions thereby defining progression periods, and select, accept or verify facts that should represent the progression period for NA assignment, i.e., select, accept or verify the best facts. In some embodiments, progression time ranges (e.g., progression periods) are suggested and facts are bucketed into those windows before suggesting a “best” fact per type, per progression with suggestions noted with computer icon. This process is referred to as enrichment herein. In some embodiments, the user has the ability to override suggestions for some or all of the element and for some or all of the progressions.

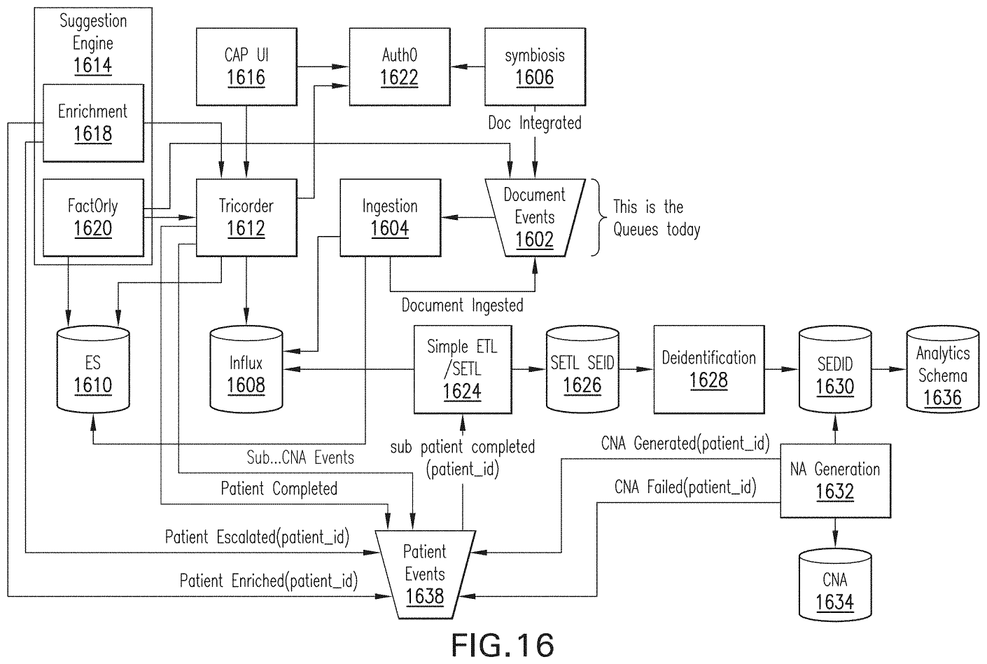

FIG. 16 is an example architecture in accordance with some embodiments. Documents including patient data (e.g., pdf, h17) and document events 1602 are ingested using a document ingestion layer, which is referred to as “symbiosis” 1606 herein. The ingested document data is stored in storage, which is labeled as “Influx” 1608 herein, that stores all data abstracted in the abstraction platform. In some embodiments, data stored in Influx 1608 is also accessed by or provided to a layer on top of the ingested document storage, which is referred to herein as “elastic search” (“ES”) 1610, that allows for searching of the ingested documents efficiently. The stored ingested documents in Influx 1608 are accessed by the abstraction platform (“AP”). In this example, “Tricorder” 1612 refers to a framework on which the AP is built. The Tricorder 1612 abstraction platform framework works with the ES 1610, a Suggestion Engine 1614, and a clinical abstraction platform user interface (CAP UI) 1616.

The Suggestion Engine 1612, which implements some aspects of the EL, performs enrichment 1618 including determining suggested progression periods and suggested best facts based on the ingested data. In some embodiments, the Suggestion Engine 1612 is part of a “decision support system” that provides input to help make decisions. In some embodiments, the Suggestion Engine 1614 also suggests abstraction values based on ingested data (e.g., HL7 data) via a process referred to as FactOrly 1620 herein. The suggested progression periods, the suggested best facts, and other candidate facts are presented to a user via a user interface, e.g., the CAP UI 1616, to receive input including input regarding acceptance or verification of the best facts or selection of other candidate facts as the best facts, and/or input regarding the suggested progression periods as indicated by “Patient Events” 1638.

If there is some conflict or issue in the patient data that needs to be addressed, data regarding the patient may be escalated and presented to a user via a user interface for resolution of the conflict or issue. If the conflict or issue cannot be resolved, the patient data may not be further processed for generation of output tables for analytics or generation of a nodal address. In some embodiments, if a patient has more than one major disease at the same time (e.g., more than one primary cancer at the same time), the patient's data may be escalated. In some embodiments, at least some patient information may be deemed essential such that if data regarding the patient data does not include the essential patient information, the system may not further process the patient data for generation of output tables for analytics or generation of a nodal address. For example, in some embodiments, if the patient data does not include a date of diagnosis, the data may not be further processed.

In some embodiments, an authentication layer, which is referred to as “AuthO” 1622 herein, is employed for secure login to the abstraction platform. The term “Extract, Transform, Load” (“ETL”) as used herein refers to the functions performed when pulling data out of one database and placing into another of a different type. ETL is used to migrate data, e.g., from relational databases (database systems in which any field can be a component of more than one of the databases) into decision support systems.

The term “relational database” as used herein refers to a database that maintains a set of separate, related files (tables), but combines data elements from the files for queries and reports when required. Routine queries to a relational database often require data from more than one file. A relational database management system has the flexibility to “join” two or more files by comparing key fields and generating a new file from the records that meet the matching criteria. In practice, a pure relational query can be very slow. To speed up the process, indexes are built and maintained on key files used for matching.

Simple ETL is a programming language based on the mathematical theory of sets, a branch of mathematics or logic concerned with sets of objections and rules for their manipulation. “Simple ETL/SETL” 1624 as used herein refers to an ETL process that transforms data stored in Influx 1608 into query-able tables organized by fact type. The SETL identified patient data output is stored in a schema referred to herein as “SETL SEID” 1626.

In some embodiments, the Simple ETL/SETL 1624 determines if there is an initial data of diagnosis associated with the patient data, and if there is no initial date of diagnosis, the data regarding the patient is not saved as SETL SEID data.

The patient data is de-identified 1628 to remove patient identification information other than an internally generated identifier associated with the patient, and the SETL de-identified patient data is stored in a schema referred to as SEDID 1630. The system attempts to assign nodal addresses to the data in a nodal address (NA) generation process 1632. The NA generation process 1632 assigns a nodal address to the patient data corresponding to a progression period. In some embodiments, the NA Generation is via a business rules engine that evaluates patient data and determines the nodal address. In some embodiments, the NA generation business rules engine includes a nodal address API service that can generate nodal address information from data sent from external sources.

The NA generation process may output information indicating that a nodal address was generated for the patient (e.g., for each progression period), or information indicating that the nodal address generation failed for the patient (e.g., for all progression periods or for at least one progression period). Nodal address generation may fail for multiple different reasons. For example, nodal address generation may fail due to insufficient information being provided for all of the different elements required for generation of a nodal address. A Nodal Address schema 1634 may be employed for the nodal address generation. The term “Nodal Address Schema” (“NA Schema”) as used herein refers to nodal address assignment metadata, including success/failure messages, historical nodal address assignment, and current database of nodal addresses.

The SETL deidentified patient data may be used by the analytics schema 1632.

In some embodiments, the analytic schema 1636 determines a treatment intent based, at least in part, on facts provided by the EL. Therapy intent requires information regarding the progression track obtained from the EL, information regarding whether the tumor is metastatic, which is from TNM derived from the time series data provided by the EL, overall stage provided by the EL, and intervention outcome within a given time window.

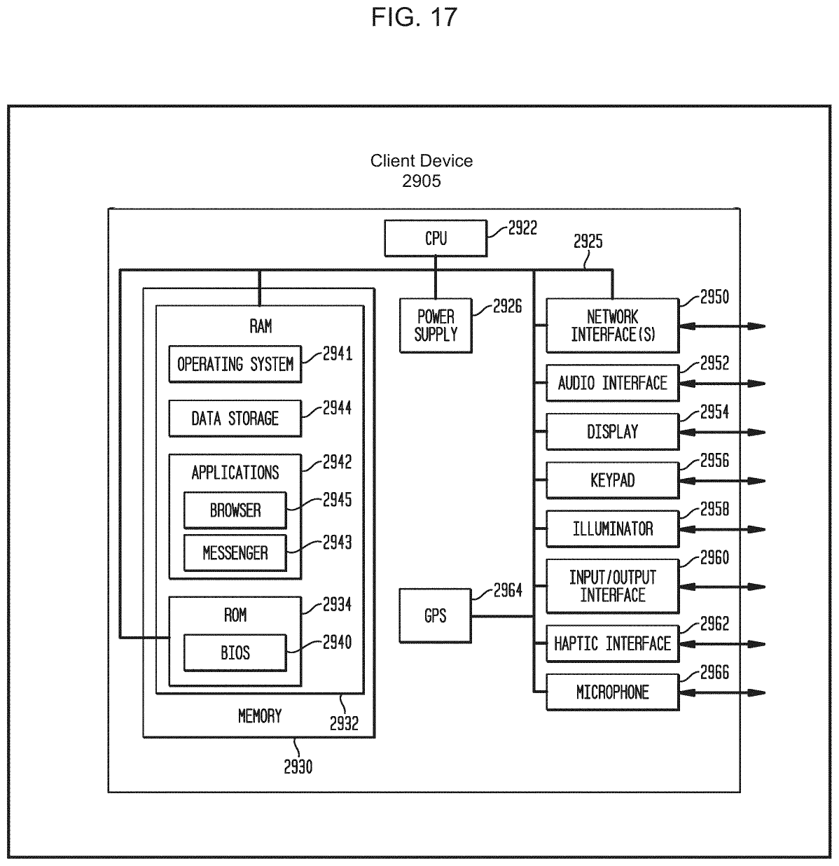

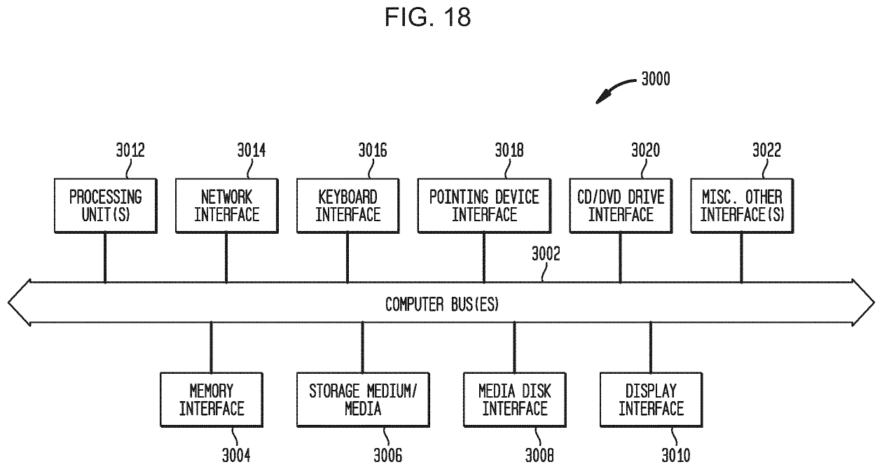

FIG. 17 is a block diagram illustrating an internal architecture of an example of a computer, such as computing system 105 and/or client computing device 110, in accordance with one or more embodiments of the present disclosure. A computer as referred to herein refers to any device with one or more processors capable of executing logic or coded instructions, and could be a server, personal computer, set top box, tablet, smart phone, pad computer or media device, to name a few such devices. As shown in the example of FIG. 18, internal architecture 3000 includes one or more processing units (also referred to herein as CPUs) 3012, which interface with at least one computer bus 3002. Also interfacing with computer bus 3002 are persistent storage medium/media 3006, network interface 3014, memory 3004, e.g., random access memory (RAM), run-time transient memory, read only memory (ROM), etc., media disk drive interface 2308 as an interface for a drive that can read and/or write to media including removable media such as floppy, CD-ROM, DVD, etc. media, display interface 3010 as interface for a monitor or other display device, keyboard interface 3016 as interface for a keyboard, pointing device interface 3018 as an interface for a mouse or other pointing device, and miscellaneous other interfaces not shown individually, such as parallel and serial port interfaces, a universal serial bus (USB) interface, and the like.

Memory 3004 interfaces with computer bus 3002 so as to provide information stored in memory 3004 to CPU 3012 during execution of software programs such as an operating system, application programs, device drivers, and software modules that comprise program code, and/or computer-executable process steps, incorporating functionality described herein, e.g., one or more of process flows described herein. CPU 3012 first loads computer-executable process steps from storage, e.g., memory 3004, storage medium/media 3006, removable media drive, and/or other storage device. CPU 3012 can then execute the stored process steps in order to execute the loaded computer-executable process steps. Stored data, e.g., data stored by a storage device, can be accessed by CPU 3012 during the execution of computer-executable process steps.

As described above, persistent storage medium/media 3006 is a computer readable storage medium(s) that can be used to store software and data, e.g., an operating system and one or more application programs. Persistent storage medium/media 3006 can also be used to store device drivers, such as one or more of a digital camera driver, monitor driver, printer driver, scanner driver, or other device drivers, web pages, content files, playlists and other files. Persistent storage medium/media 3006 can further include program modules and data files used to implement one or more embodiments of the present disclosure.

Internal architecture 3000 of the computer can include (as stated above), a microphone, video camera, TV/radio tuner, audio/video capture card, sound card, analog audio input with A/D converter, modem, digital media input (HDMI, optical link), digital I/O ports (RS232, USB, FireWire, Thunderbolt), and/or expansion slots (PCMCIA, ExpressCard, PCI, PCIe).

Reviewing relevant information from an electronic medical record for diagnostic or treatment purposes or for a visit with a patient can require significant amounts of time for a medical provider. Further, many interfaces for viewing information from an electronic medical record only display certain aspects of the patient's medical information at one time, or only certain date ranges for a patient's medical information at one time, or require clicking through multiple menus to determine if there is any relevant medical information of a certain type, which can lead to relevant medical information being easily overlooked. Some embodiments provide a graphical user interface including an interactive timeline for viewing information regarding a patient's medical history that provides an overview of relevant medical information (e.g., diagnostic information, treatment information, biomarker information, disease progression information) and efficient access to detailed medical information at the same time. In some embodiments, such a graphical user interface with an interactive patient timeline can be used by a medical provider to review a patient's medical history upon intake, before or during a patient visit, or prior to seeing a patient in an emergency room visit. In some embodiments, such an interactive patient timeline can be used by a medical provider in a handoff between modalities, e.g., between a primary care physician and a specialist, or by a tumor board. In some embodiments, the interactive patient timeline can be used for review of a patient's medical history instead of accessing a patient's electronic medical record.

In some embodiments, the interactive patient timeline is generated from time series data, which is described above. In some embodiments, the interactive patient timeline is based on all relevant non-duplicative patient data or information and not just best fact data. In some embodiments, the interactive patient timeline includes an indication of best fact data. In some embodiments, providing the interactive patient timeline includes grouping some facts as associated with a disease progression. In some embodiments, providing the interactive patient timeline includes determining a span or duration of time associated with medical information based on the patient data.

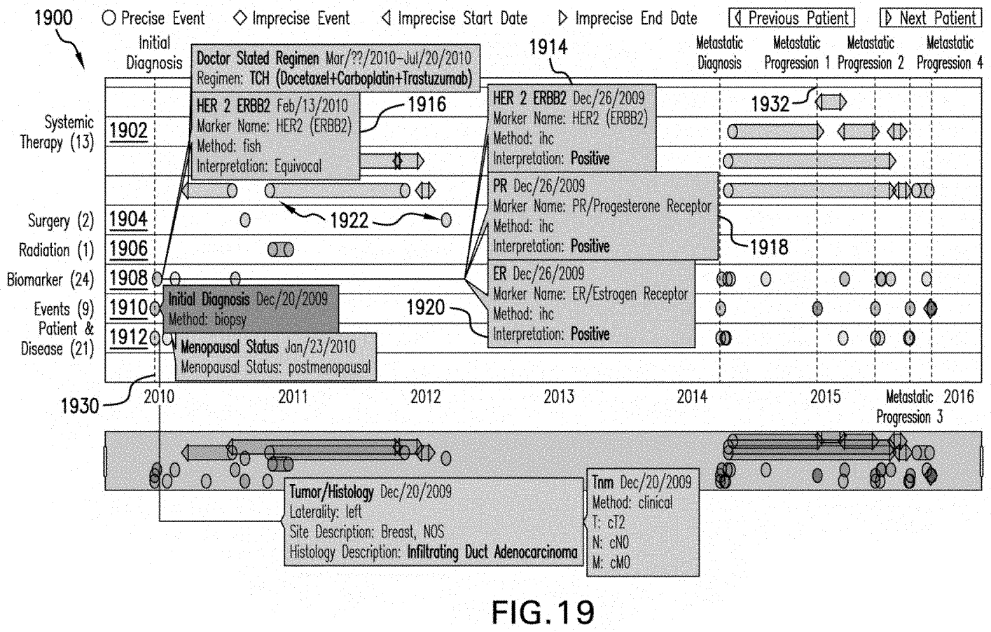

FIGS. 19-24 illustrate a graphical user interface including an interactive patient timeline in accordance with some embodiments. The timelines may be generated by a computing system (e.g., computing system 105) and displayed on a GUI (e.g., GUI 150a, 150b) in accordance with an exemplary embodiment. In some embodiments, generating of the graphical user interface including an interactive patient timeline may be based on a browser-based graphing library, such as plotly for Python.

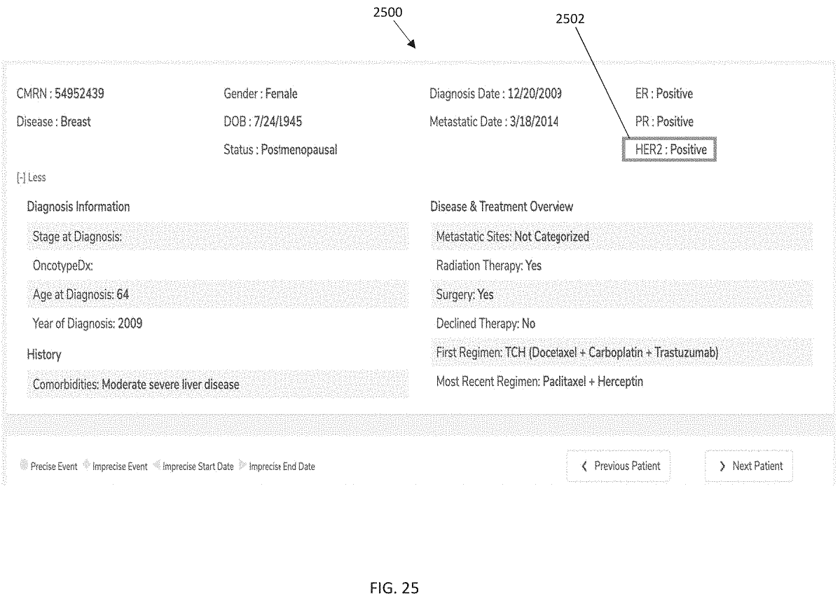

FIG. 19 illustrates an example interactive patient timeline 1900 in accordance with an exemplary embodiment. Medical information of the patient (information in the patient's medical history or patient's medical record) shown in the patient timeline in FIGS. 19-24 and in FIG. 25 is not real patient data, but is instead mock data based on a common clinical scenario that a clinician would encounter in practice. The patient interactive timeline 1900 includes a plurality of markers (e.g., marker 1922) which are displayed as a circle, triangle, diamond, or other shape on the timeline. Each marker indicating a relevant time associated with medical information, the beginning of a period of time associated with medical information, or the end of a period of time associated with medical information. Each time period associated with medical information is graphically displayed with a beginning marker and an ending marker and a graphical indication of span between the beginning marker and the ending marker.

A user selection of a marker causes the timeline to display medical information associated with the marker. For example, a user may employ a mouse, touch pad or touch sensitive screen to move a cursor to select a marker and view a display of medical information associated with the selected marker. In one embodiment, the user may use the cursor to hover over a marker, resulting in a graphical window associated with the marker to pop up. In some embodiments, the interactive timeline may include a plurality of sub-timelines that are vertically offset and aligned in time with each other for different categories of information. In some embodiments, the plurality of sub-timelines includes one or more of: a treatment sub-timeline including any markers related to treatment information (e.g., Systemic Therapy sub-timeline 1901, Surgery sub-timeline 1904, or Radiation sub-timeline 1906); a diagnosis or progression sub-timeline including any markers related to diagnosis, or disease or disorder progression information (e.g., Events sub-timeline 1910); a biomarker sub-timeline 1908 including any markers related to disease or disorder biomarker test results information (e.g., Biomarker sub-timeline 1908); a disease or disorder sub-timeline including markers related to disease or disorder information not falling in other categories (e.g., Patient & Disease timeline 1912); and a patient sub-timeline including any markers related to relevant patient information not falling into other categories (e.g., Patient & Disease timeline 1912). As shown in FIG. 19, in some embodiments the interactive timeline includes sub-timelines corresponding to Systemic Therapy 1902, Surgery 1904, Radiation 1906, Biomarker information 1908, Diagnosis or Progression 1910, and Patient and Disease information 1912. Markers in each sub-timeline may be displayed in different colors in some embodiments. Each sub-timeline may display markers graphically representing medical information in chronological order associated with that sub-timeline category. For example, the Biomarker sub-timeline 1908 includes markers associated with biomarker testing results for the patient. Upon selection of a marker by a user, information is displayed regarding the medical information regarding the marker. For example, receipt of a user selection of a marker within the biomarker sub-timeline 1908 may display information including one or more of the date of the test, the name of the biomarker that is tested for (e.g., HER2, Progesterone Receptor, Estrogen Receptor, etc.), the method of testing (e.g., FISH, MC, etc.), the results, and the interpretation, It should be appreciated that different embodiments may include different timeline categories.

In some embodiments, one or more vertical graphical indicators are used to represent a diagnosis or a progression of a disease or disorder. For example, in FIG. 19, vertical graphical indicator 1930 corresponds to the time of initial diagnosis, and vertical graphical indicator 1932 corresponds to a first metastatic progression. In some embodiments, the interactive timeline includes one or more diagnosis or progression time periods. In some embodiments, the one or more diagnosis or progression time periods are divided by the one or more vertical graphical indicators. In some embodiments, the interactive timeline includes one or more diagnosis or progression time periods. In some embodiments, the graphical user interface enables filtering of markers displayed the interactive timeline based on user-selected criteria. In some embodiments, the user-selected criteria include a diagnosis or progression time period.

As noted above, in some embodiments, the interactive timeline includes markers corresponding to relevant non-duplicative information and is not limited to just the determined best fact information. For example, the patient timeline 1900 shows a breast cancer patient who had received two HER2 tests when the patient was diagnosed non-metastatic—selection of one marker 1914 displays information regarding a ‘Positive’ IHC test performed on Dec. 26, 2009, and selection of another marker 1916 displays information regarding an ‘Equivocal’ FISH test performed on Feb. 13, 2010.

It should also be appreciated that the described sub-timelines of FIGS. 19-24, including timeline 1900, may display additional markers besides those discussed in relation to HER2 testing. For example, in association with the Biomarker sub-timeline 1908, the timeline 1900 includes a marker 1918 with an associated window displaying that the patient received a Progesterone Receptor test with a ‘Positive’ IHC test on Dec. 26, 2009, and a marker 1920 with an associated window displaying that the patient received an Estrogen Receptor test with a ‘Positive’ IHC test on Dec. 26, 2009. The timeline 2100 further displays markers associated with the Systemic Therapy sub-timeline 1902, the Events sub-timeline 1910, and the Patient and Disease sub-timeline 1912.

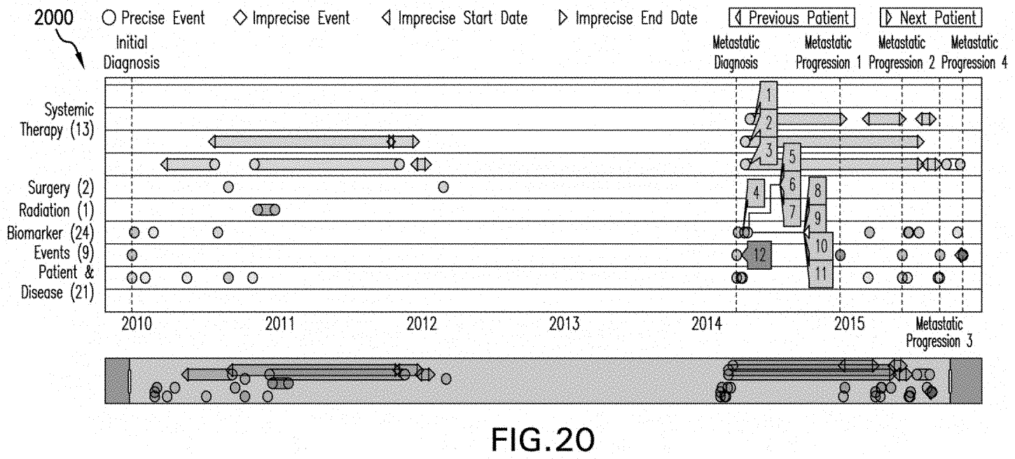

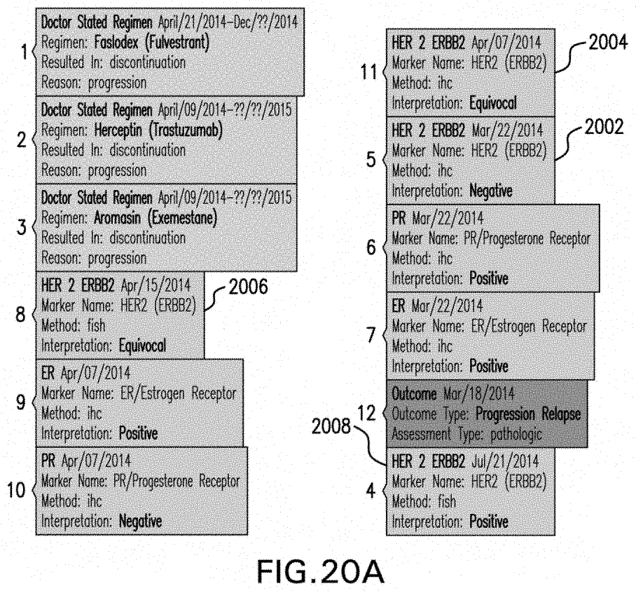

FIG. 20 illustrates the timeline 2000 in accordance with an exemplary embodiment with markers associated with the first progression after diagnosis selected. The view of the interactive patient timeline 2000 in FIG. 20 shows that the patient received four additional HER2 tests after the first disease progression when the patient was diagnosed metastatic: selected marker 2002 displays a ‘Negative’ IHC test performed on Mar. 22, 2014, selected marker 2004 displays an ‘Equivocal’ IHC test performed on Apr. 7, 2014, selected marker 2006 displays an ‘Equivocal’ FISH test performed on Apr. 15, 2014, and selected marker 2008 displays a ‘Positive’ FISH test performed on Jul. 21, 2014.

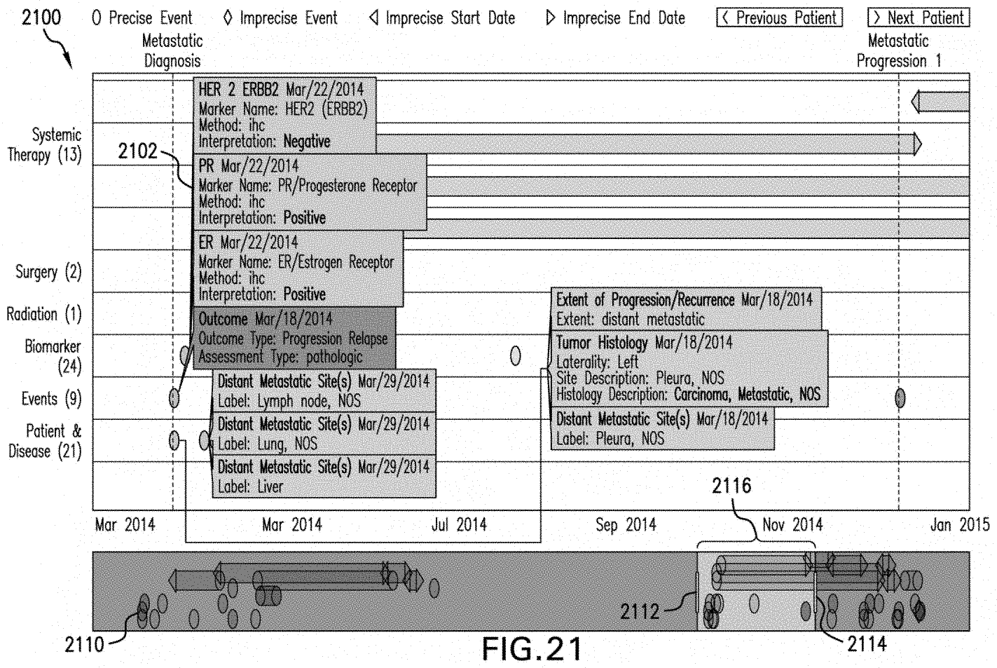

In some embodiments, the method further comprises displaying a summary version of the full time period timeline 2110 including two or more selectable graphical indicators, the selectable graphical indicators including a beginning time period indicator 2112 and an ending time period indicator 2114, where user selection and movement of the beginning time period indicator and/or the ending time period indicator change a time period 2116 displayed in the interactive timeline. This is illustrated in FIG. 21, which depicts a zoomed in view of a time period including the progression to metastatic cancer in the interactive patient timeline with the smaller summary version of the full time period timeline below showing the period selected. The view in FIG. 21 provides display information regarding the selected marker 2102 corresponding to information regarding a “Negative” HER2 IHC test conducted on Mar. 22, 2014 post-progression to metastatic cancer.

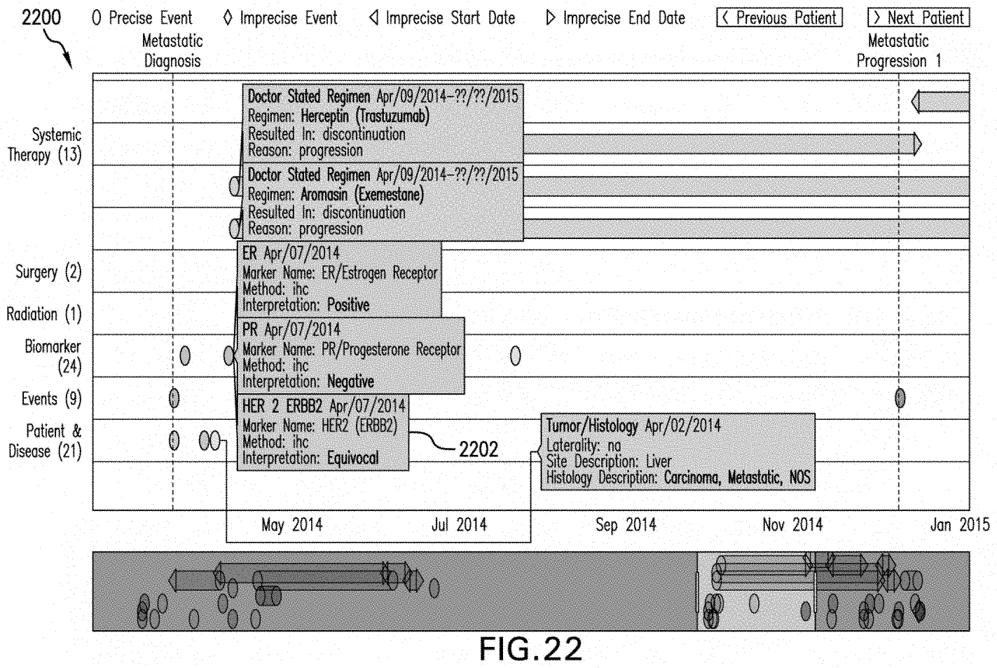

FIG. 22 illustrates another zoomed in view of a portion of the interactive timeline with displayed information regarding selected marker 2202 showing an “Equivocal” result of a second HER2 IHC test conducted on Apr. 7, 2014 post-progression to metastatic cancer.

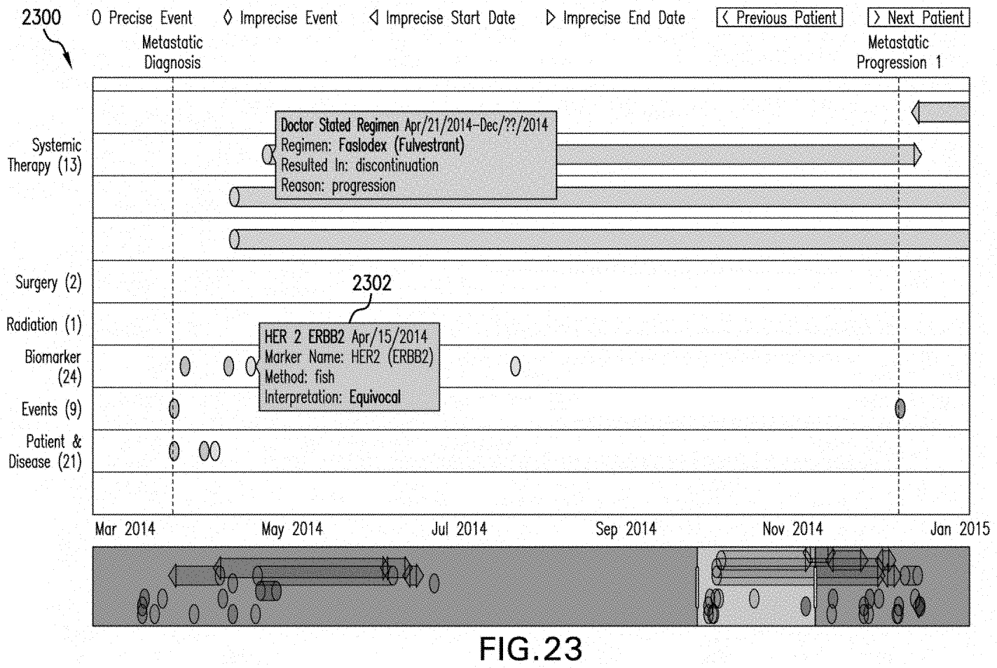

In FIG. 23, user selection of the 2302 marker causes display of information regarding an “Equivocal” result of a third HER2 test, which was a FISH test, conducted on Apr. 15, 2014 post-progression to metastatic cancer.

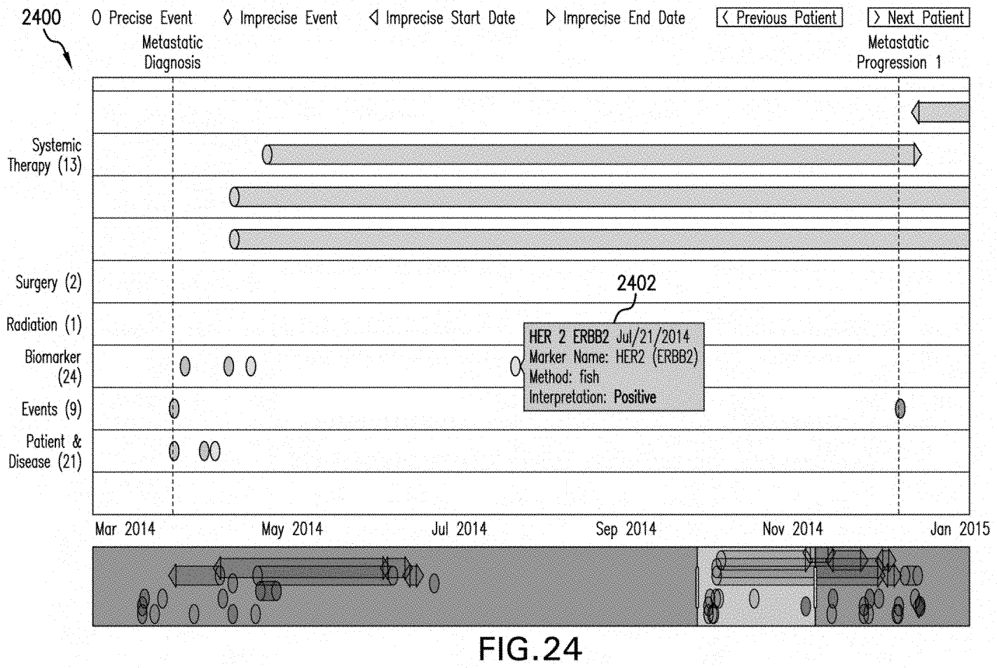

In FIG. 24, user selection of the 2402 marker causes display of information regarding a “Positive” result of a fourth HER2 test, which was a FISH test, conducted on Jul. 21, 2014 post-progression to metastatic cancer.

As described in the present disclosure, the method or system can evaluate end to end patient information covering the course of the patient's medical history from diagnosis through multiple points up until death and generate suggestions for the most accurate facts regarding the patient from the patient information, subject to acceptance or verification. In some embodiments, the system or method generates the timeline(s) based output representing the identified best facts after acceptance or verification. The best facts corresponding to each element associated with the patient can represent a complete and current view of the patient's medical condition and illness history.

Although a clinician may want to view all relevant non-duplicative information from a patient's medical record in a patient timeline, the patient timeline depicted in FIGS. 19-23 illustrates some challenges associated with patient records containing potentially conflicting information. For example, the patient whose medical history was depicted in FIGS. 19-23 had received a total of six HER2 tests (2 while non-metastatic, 4 while metastatic) in the course of her clinical history. To determine and assign a correct HER2 status, a clinical and data team would have to implement sophisticated rules and logic to determine the best and correct HER2 status at the point of query. For this example, based on timing and test reliability and accuracy, this patient is determined to be HER2 positive at the time of developing metastatic disease due to the best fact according to the reduction rules, i.e., the positive FISH test.

In some embodiments, to determine and correctly assign HER2 status is the function of the best fact selection feature. In some embodiments, best fact selection determines the patient's HER2 status at the time of metastatic diagnosis. Although the interactive timeline may not be limited to best facts in some embodiments, the best facts may be used for determination of associated summary information regarding the patient, such as HER2 positive status. FIG. 25 illustrates an example interface 2500 displaying a summary associated with the patient in accordance with an exemplary embodiment. The summary information for the patient of FIGS. 19-24 indicates the patient's HER2 status.

The identification of best facts for patients simplifies population-level metric aggregation in some embodiments. For example, an institution may inquire regarding how many first-line metastatic, HER2 positive patients were seen by the institution within a particular year. This is not a trivial question to answer given the potential discrepancy that may exist in HER2 testing data in a patient's medical record. In some embodiments, the method or system of the present disclosure enables a data team or technology to systemically assign HER2 status across all patients for the purpose of fulfilling data requests or answering clinical questions. Furthermore, accurate testing of HER2 status is of great importance for patients in order to provide the best treatment for patients with metastatic disease.

In some embodiments a patient timeline may include only best facts. In some embodiments timelines for multiple different patients may be compared with timelines for different patients overlaid with respect to diagnosis or one or more progressions.

In some embodiments, an analytical system or method may show a de-identified, longitudinal and comparable patient journey or timeline which shows markers corresponding to clinical significant medical information and can be used as a tool for retrospective analysis of patient journeys (as a research cohort) to inform physicians of future directions they may be able to take with their own patient population. The timeline will still be clinically relevant, with the events shifted during de-identification in a way such that the physician can still use it for research or other purposes. The timeline will highlight disease progressions, which will help illuminate the causes of that progression. In some embodiments, an overlay of each progression of the disease will then help medical providers identify options that exist for treatments that may or may not have been considered, based on treatments offered to similar patients and their associated outcomes.

FIG. 26 illustrates an example interface 2600 displaying patient information for analysis for an institution or an organization in accordance with an exemplary embodiment. The interface 2600 may be displayed on a GUI (e.g., GUI 150a, 150b) in accordance with an exemplary embodiment. As shown in interface 2600, within a dataset of 8,106 patients (2602), there are 203 patients (2604) who were HER2 positive at the time of their first line therapy for metastatic breast cancer. Aggregating such information regarding patients requires determining a definitive HER2 status for each patient at each progression, which can be implemented via the best facts methods and systems described herein. Thus, the described method or system can use the best fact identification to determine how many first-line metastatic, HER2 positive patients that were seen by the institution within a particular year.

In some embodiments, systems and methods that employ enrichment as described herein may generate best facts for use by analytical systems, programs, or apps to analyze patient data, or are incorporated into systems and methods that employ analytical systems. For example, the Real Word Analytics web application from COTA, Inc. (AKA COTA Healthcare) is a population health analytics tool that can employ best facts selection and provide a summary of diagnostics, procedures, treatments, and outcomes so a healthcare administrator's clinical team can retrospectively uncover insights in similar patient cohorts. An analytics program, system or app, using the best facts selection can also or alternatively be used to track key operational metrics and clinical insights and the ability to investigate outliers to better understand aggregate metrics in some embodiments. The data provided to analytical systems may be de-identified as to patient to ensure patient privacy. In some embodiments, such a system, method or application, may have a user-interface similar to that depicted in FIG. 26 for Real World Analytics (RWA).

In some embodiments, enrichment as described herein is employed in a system or method that summarizes clinically relevant attributes of a patient population, including, but not limited to, tumor histology, stage, comorbidities, outcomes, and therapies including surgery, radiation, and chemotherapy. In some embodiments, enrichment as described herein is employed in a system or method that tracks and enables retrospective analysis of operational metrics including, but not limited to, patients under treatment, metastases, molecular markers, progression-free and overall survival, additional cancer-specific diagnostic attributes, and clinical population. In some embodiments, enrichment as described herein is employed in a system or method that enables selection and filtering of patient cohorts and sub-cohorts based on cancer-specific diagnostic attributes (e.g., breast cancer-specific diagnostic attributes, lung cancer-specific diagnostic attributes) and cohort comparison tools. In some embodiments, the patient cohorts and sub-cohorts are determined at least in part, based on progression obtained from best fact data or best fact selection as described herein. In some embodiments, the patient cohorts and sub-cohorts are determined at least in part, based on a nodal address or nodal addresses to which the patients are assigned, where the nodal address or nodal addresses are assigned based, at least in part, on best fact data or facts determined from best fact selection as described herein. In some embodiments, enrichment as described herein is employed in a system or method that enables aggregation and visualization of treatment choices by practice site and physician. In some embodiments, enrichment as described herein is employed in a system or method that compares treatments and regimen combinations, including their sequencing. In some embodiments, enrichment as described herein is employed in a system or method that visualizes a treatment journey of patient at multiple levels of granularity (e.g., line of therapy, modalities involved). FIG. 27 is a schematic diagram for a system and method, in which enrichment and best fact selection is incorporated, that produces enriched longitudinal patient records and best facts per progression, as well as provides analytics and tools (e.g., such as the analytics and tools provided in COTA RWA) for the use of health providers, health systems, for medical system management, for those who authorize care or payment for care, for those who evaluate care, and/or for other users in accordance with some embodiments. The system and method can be implemented, at least in part, via a web-based application in some embodiments. In some embodiments, the system and method include an Application Front End 2710, which may be formed as part of a web-interface for a user. In some embodiments, a Cloud Container Engine 2720 may implement an API, and may incorporate some or all of data templates, data tables and pre-aggregations that power the application, survival and metrics 2722. Information from Cloud Container Engine 2720 is provided to a Data Cache 2730, to User Settings 2732, and to Data Insights 2734. Data Insights 2734 also receives input from an Insights Scheduler 2736. Data Insights 2734 saves queries by the application for all users, which are stored, and calculates the patient count to perform analytics based on query criteria at regular time intervals based on the Insights Scheduler 2736. User Settings 2732 are saved queries for the current user only. In some embodiments, input medical history data or medical record data for patients is not received via the same Application Front End, but is instead obtained separately. In some embodiments, the data is abstracted from records and is input as Time Series Patient Facts 2738. The Time Series Patient Facts 2738 undergo Enrichment / Best Fact Selection 2740, as described herein. Nodal Address (NA) and Progression Assignment 2742 is conducted based on the input data after the enrichment and best fact selection. After NA and Progression Assignment 2742, in some embodiments Data Transformations 2744 are employed. Data transformations include additional rulesets for data such as, but not limited to, treatment sequencing, combinations, timeline segments, etc. The transformed data is used along with data from the Data Cache 2730 and Data Insights 2734 to produce Enriched Longitudinal Patient Records and Best Facts per progression 2746. In some embodiments, the Enriched Longitudinal Patient Records and Best Facts per progression are stored remote from a user at a server or a cloud environment and controlled by a provider of the web-based application. The Enriched Longitudinal Patient Records and Best Facts per progression are then used in analytics, e.g., to determine cohorts with the same or similar disease-relevant characteristics in one or more progression periods to compare like patients to like patients. The analytics are performed in/by the Cloud Container Engine 2720. In some embodiments, the system or method enables the user to export at least some of the enriched longitudinal patient records and best facts data. In some embodiments, a method or system employs user privileges to determine whether any of the enriched longitudinal patient records and facts data can be exported by a user.

In some embodiments, the best fact data is stored primarily within the progression based data. In some embodiments, best facts data can be joined to times series data using a unique patient identifier.

In some embodiments of systems and methods, enriched longitudinal patient records and/or best facts per progression are used to define a disease-relevant cohort of patients each having the same parameters for disease-relevant characteristics for a particular disease (e.g., patients assigned to the same or a closely related nodal address during one or more progression periods) to conduct analysis, compare outcomes, and/or perform outcome tracking. In some embodiments, the outcome tracking and/or comparison of outcomes enables identification of whether a patient in the cohort is experiencing worse outcomes than expected based on the outcomes for other patients having the same parameters for disease-relevant characteristics. In some embodiments, the outcome tracking or comparison of outcomes enables identification of whether one or more patients in the cohort are experiencing worse outcomes than expected based on the outcome tracking or comparison of outcomes for other patients or all having the same parameters for disease-relevant characteristics in the cohort (e.g., all patients assigned to the same or a closely related nodal address during one or more progression periods). In some embodiments, the outcome tracking or outcome comparison enables identification of whether one or more patients in the cohort being cared for by a particular provider, group, or site are experiencing worse outcomes than expected based on the outcome tracking or outcome comparison for patients having the same parameters for disease-relevant characteristics (e.g., all patients assigned to the same or a closely related nodal address during one or more progression periods) being cared for by other providers, groups, or at other sites. In some embodiments, the outcome tracking or outcome comparison enables an alert, a communication to a health care provider, or visual indication that the patient or patients is/are experiencing worse outcomes than expected based on the outcome tracking, enabling the health care provider to take corrective action. Further information regarding outcome tracking, alerts, and communications can be found, at least, in U.S. Published Patent Application No. 2015/0100341, U.S. Patent Application Publication No. US 2021/0082573, and International Patent Application Publication No. WO 2018/089584, each of which is incorporated by reference herein in its entirety.

In some embodiments of systems and methods, enriched longitudinal patient records and/or best facts per progression are used to define a disease-relevant cohort of patients each having the same parameters for disease-relevant characteristics for a particular disease (e.g., patients assigned to the same or a closely related nodal address during one or more progression periods) in a decision support system or method that aids in determining potentially effective and efficient treatment options for a patient (e.g., a bundle of patient treatment services associated with a nodal address to which a patient is assigned). In some embodiments, enriched longitudinal patient records and/or best facts per progression are used to define a disease-relevant cohort of patients each having the same parameters for disease-relevant characteristics for a particular disease (e.g., patients assigned to the same or a closely related nodal address during one or more progression periods) in a system or method for comparison of treatments and outcomes, which can be used by the system or method to guide treatment of or provide suggested treatment options for a patient assigned to the same nodal address. Further information regarding providing treatment options for patients may be found, at least, in U.S. Published Patent Application No. 2015/0100341, U.S. Patent Application Publication No. US 2021/0082573, and International Patent Application Publication No. WO 2018/089584, each of which is incorporated by reference herein in its entirety.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the user computing device or server or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter. While the system and method have been described in terms of one or more embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method for providing accurate patient data for a patient with a medical condition and/or illness, the method comprising:
accessing an initial set of data records associated with the patient, the initial set of data records including information regarding the patient, the patient's illness, and/or the patient's treatment;
extracting a plurality of candidate facts from the accessed initial set of data records, each candidate fact represented as a data set;
categorizing each candidate fact as corresponding to an element of a plurality of elements associated with the patient, the plurality of candidate facts including more than one candidate fact corresponding to the element for at least one element in the plurality of elements;
for elements that are unchanging over time, identifying at least one best fact corresponding to each element, the identifying including:
where the element has only one corresponding candidate fact, identifying the corresponding candidate fact as the best fact corresponding to the element; and
where the element has at least two corresponding candidate facts, identifying at least one of the corresponding candidate facts for the element as the best fact for the element based on reduction rules specific to the element;
for each element that can change over time, associating each candidate fact corresponding to the element with progression period corresponding to a diagnosis or progression milestone;
for each element that can change over time, identifying at least one best fact for each progression period having an associated candidate fact for the element, the identifying including:
where the element has only one corresponding candidate fact associated with the progression period, identifying the corresponding candidate fact as the best fact corresponding to the element for the progression period; and
where the element has at least two corresponding candidate facts associated with the progression period, identifying at least one best fact corresponding to the element for the progression period from the at least two corresponding candidate facts based on reduction rules specific to the element; and
outputting data including the best facts associated with the patient.

2. The method of claim 1, wherein, for at least some of the elements that are unchanging over time, identifying the at least one best fact corresponding to the element further comprises:
presenting the at least one best fact as a suggested at least one best fact corresponding to the element to a user via a graphical user interface;
receiving one or more of:
an acceptance of the suggested at least one best fact;
an identification of at least one other candidate fact that is not a suggested best fact as at least one best fact; and
a rejection of the suggested at least one best fact as a best fact; and
where a rejection of the suggested at least one best fact is received, no longer identifying the suggested at least one best fact as a best fact corresponding to the element,
where an acceptance of the suggested at least one best fact is received, identifying the at least one best fact an accepted best fact;
where an identification of at least one other candidate fact that is not a suggested best fact as the at least one best fact is received, identifying the at least one other candidate fact as the at least one accepted best fact; and
wherein outputting data regarding the best facts associated with the patient comprises outputting data regarding the accepted best facts associated with the patient.

3. The method of claim 1, wherein, for at least some of the elements that can change over time, identifying at least one best fact for each progression period having an associated candidate fact for the element further comprises:
presenting the at least one best fact for the progression period as a suggested at least one best fact corresponding to the element;
receiving one or more of:
an acceptance of the suggested at least one best fact as at least one best fact;
an identification of at least one other candidate fact that is not a suggested best fact as at least one best fact; and
a rejection of the suggested at least one best fact as a best fact; and
where a rejection of the suggested at least one best fact is received, no longer identifying the suggest at least one best fact as a best fact corresponding to the element.

4. The method of claim 1, wherein the output data including the best facts associated with the patient includes one or both of a time series output and a progression output.

5. The method of claim 4, wherein the progression output includes the best facts stored in associated concept tables, each concept table including a progression track identifier and a patient identifier; or
wherein the time series output includes the best facts stored in associated concept tables, each associated concept table indexed by a function of time elapsed between a start date and time associated with the best fact in the associated concept table.

6. The method of claim 1, further comprising:
determining, based on at least some of the candidate facts, one or more progression periods, each progression period corresponding to a period of time beginning at diagnosis or at a progression of the medical condition or illness and ending at a next progression, at the present time, or at death; and
assigning each candidate fact to a progression period.

7. The method of claim 6, further comprising:
presenting the determined one or more progression periods to a user via a graphical user interface as suggested progression periods;
receiving input from a user including one or more of:
an acceptance of at least one of the one or more suggested progression periods;
an adjustment of a start time or an end time of at least one of the one or more suggested progression periods;
an addition of a new progression period; or
merging of at least some of the one or more of the suggested progression periods in to a single progression time period; and
adjusting the one or more progression periods based on the received input, wherein each candidate fact is assigned to a progression time period after the adjusting.

8. The method of claim 6, wherein the progressions correspond to one or more of:
a physician's identification that the patient's disease or condition has progressed;
a measured growth of a tumor of the patient;
an indication that the patient's disease has spread and become metastatic;
an indication that the patient's disease or medical condition has not responded to a course of treatment and a physician has decided to switch to a different course of treatment; or
an indication that the patient has experienced a relapse in disease or the medical condition.

9. The method of claim 1, wherein, for each element that can change over time, the associating of each candidate fact corresponding to the element with a progression period is based on time windowing.

10. The method of claim 1, further comprising:
accessing a new set of data records;
extracting additional candidate facts, each of the additional candidate facts corresponding to an element of the plurality of elements associated with the patient; and
determining one or more best facts corresponding to the each element of the plurality of elements based on the plurality of candidate facts extracted from the initial set of data records and the additional candidate facts extracted from the new set of data records.

11. The method of claim 1, further comprising de-duplicating the plurality of candidate facts by, for each element in the plurality of elements, removing each duplicative candidate fact.

12. The method of claim 1, further comprising:
deriving a candidate fact for at least one element of the plurality of elements associated with the patient based on one or more of the candidate facts extracted from the data and one or more medical rules.

13. The method of claim 1, wherein, for at least one of the elements, the reduction rules include one or more of:
a rule to identify at least one candidate fact as the best fact corresponding to an element based the at least one candidate fact including the most amount of data as compared to other candidate facts corresponding to the same element;
a rule to discard a candidate fact that is duplicative of and identical to another candidate fact corresponding to an element for a progression period; and
a rule to identify a candidate fact as a best fact based, at least in part, on the candidate fact being the most frequently occurring as compared to other candidate facts corresponding to the same element.

14. The method of claim 1, further comprising:
for at least one progression period, generating a nodal address for the progression period for the patient based on the output data.

15. The method of claim 14, further comprising:
providing predetermined treatment plan information to a health care provider of the patient for facilitation of treatment decisions, the predetermined treatment plan information based on the nodal address for the progression period assigned to the patient.

16. The method of claim 14, further comprising:
determining a prognosis-related expected outcome with respect to occurrence of the defined end point event for the patient based on the nodal address for the progression period assigned to the patient.

17. The method of claim 14, wherein the nodal address is a refined nodal address.

18. A system for providing accurate patient data for a patient with a medical condition and/or illness, the method comprising:
one or more data repositories; and
a computing system in communication with the one or more data repositories and configured to execute instructions that when executed cause the computing system to:
access from the one or more data repositories, an initial set of data records associated with the patient, the initial set of data records including information regarding the patient, the patient's illness, and/or the patient's treatment;
extract a plurality of candidate facts from the accessed initial set of data records, each candidate fact represented as a data set;
categorize each candidate fact as corresponding to an element of a plurality of elements associated with the patient, the plurality of candidate facts including more than one candidate fact corresponding to the element for at least one element in the plurality of elements;
for elements that are unchanging over time, identify at least one best fact corresponding to each element, the identification including:
where the element has only one corresponding candidate fact, identifying the corresponding candidate fact as the best fact; and
where the element has at least two corresponding candidate facts, identifying at least one of the corresponding candidate facts for the element as the best fact for the element based on reduction rules specific to the element;
for each element that can change over time, associate each candidate fact corresponding to the element with progression period corresponding to a diagnosis or progression milestone;
for each element that can change over time, identify at least one best fact for each progression period having an associated candidate fact for the element, the identification including:
where the element has only one corresponding candidate fact associated with the milestone, identifying the corresponding candidate fact as the best fact corresponding to the element for the progression period; and
where the element has at least two corresponding candidate facts associated with progression period, identifying at least one best fact corresponding to the element for the milestone from the at least two corresponding candidate facts based on reduction rules specific to the element; and
output data including the best facts associated with the patient.

19. The system of claim 18, wherein for at least some of the elements that are unchanging over time, identification of the at least one best fact corresponding to the element further comprises:
presenting the at least one best fact as a suggested at least one best fact corresponding to the element to a user via a graphical user interface;
receiving one or more of:
an acceptance of the suggested at least one best fact;
an identification of at least one other candidate fact that is not a suggested best fact as at least one best fact; and
a rejection of the suggested at least one best fact as a best fact; and
where a rejection of the suggested at least one best fact is received, no longer identifying the suggested at least one best fact as a best fact corresponding to the element,
where an acceptance of the suggested at least one best fact is received, identifying the at least one best fact an accepted best fact;
where an identification of at least one other candidate fact that is not a suggested best fact as the at least one best fact is received, identifying the at least one other candidate fact as the at least one accepted best fact; and
wherein the output data regarding the best facts associated with the patient comprises output regarding the accepted best facts associated with the patient.

20. The system of claim 18, wherein, for at least some of the elements that can change over time, the identification of at least one best fact for each progression period having an associated candidate fact for the element further comprises:
presenting the at least one best fact for the progression period as a suggested at least one best fact corresponding to the element;
receiving one or more of:
an acceptance of the suggested at least one best fact as at least one best fact;
an identification of at least one other candidate fact that is not a suggested best fact as at least one best fact; and
a rejection of the suggested at least one best fact as a best fact; and
where a rejection of the suggested at least one best fact is received, no longer identifying the suggest at least one best fact as a best fact corresponding to the element.

21. The system of claim 18, wherein the output data including the best facts associated with the patient includes one or both of a progression output and a time series output.

22. The system of claim 21, wherein:
the progression output includes the best facts stored in associated concept tables, each concept table including a progression track identifier and a patient identifier; or
the time series output includes the best facts stored in associated concept tables, each associated concept table indexed by a function of time elapsed between a start date and time associated with the best fact in the associated concept table, or
both.

23. The system of claim 18, wherein the instructions, when executed, further cause the computing system to:
determine, based on at least some of the candidate facts, one or more progression periods, each progression period corresponding to a period of time beginning at diagnosis or at a progression of the medical condition or illness and ending at a next progression, at the present time, or at death; and
assign each candidate fact to a progression period.

24. The system of claim 23, wherein the instructions, when executed, further cause the computing system to:
present the determined one or more progression periods to a user via a graphical user interface as suggested progression periods;
receive input from a user including one or more of:
an acceptance of at least one of the one or more suggested progression periods;
an adjustment of a start time or an end time of at least one of the one or more suggested progression periods;
an addition of a new progression period; or
merging of at least some of the one or more of the suggested progression periods in to a single progression time period; and
adjust the one or more progression periods based on the received input, wherein each candidate fact is assigned to a progression time period after the adjustment.

25. The system of claim 23, wherein the progressions correspond to one or more of:
a physician's identification that the patient's disease or condition has progressed;
a measured growth of a tumor of the patient;
an indication that the patient's disease has spread and become metastatic;
an indication that the patient's disease or medical condition has not responded to a course of treatment and a physician has decided to switch to a different course of treatment; or
an indication that the patient has experienced a relapse in disease or the medical condition.

26. The system of claim 23, wherein, for each element that can change over time, the association of each candidate fact corresponding to the element with a progression period is based on time windowing.

27. The system of claim 18, wherein the instructions, when executed, further cause the computing system to:
access a new set of data records;
extract additional candidate facts, each of the additional candidate facts corresponding to an element of the plurality of elements associated with the patient; and
determine one or more best facts corresponding to the each element of the plurality of elements based on the plurality of candidate facts extracted from the initial set of data records and the additional candidate facts extracted from the new set of data records.

28. The system of claim 18, wherein the instructions, when executed, further cause the computing system to do one or more of:
de-duplicate the plurality of candidate facts by, for each element in the plurality of elements, removing each duplicative candidate fact;
derive a candidate fact for at least one element of the plurality of elements associated with the patient based on one or more of the candidate facts extracted from the data and one or more medical rules.

29. The system of claim 23, wherein the instructions, when executed, further cause the computing system to:
for at least one progression period, generate a nodal address for the progression period for the patient based on the output data.

30. A method for providing a graphical user interface for visualizing patient data, the method comprising:
displaying an interactive timeline graphically depicting information regarding a patient's medical history, the interactive timeline including a plurality of markers each marker indicating a relevant time associated with medical information, a beginning of a period of time associated with medical information, or an end of a period of time associated with medical information, the interactive timeline including a plurality of sub-timelines for different categories of patient information vertically offset and aligned in time with each other, the plurality of sub-timelines including one or more of: a treatment sub-timeline including any markers related to treatment information, a diagnosis or progression sub-timeline including any markers related to diagnosis or disease or disorder progression information, a biomarker sub-timeline including any markers related to disease or disorder biomarker test results information, a disease or disorder sub-timeline including any markers related to disease or disorder information not falling in other categories, and a patient sub-timeline including any markers related to relevant patient information not falling into other categories;
receiving a user input selecting a marker; and
displaying detailed medical information associated with the marker in a window in the interactive timeline.
